(19) United States
(12) Patent Application Publication
(10) Pub. No.: US 2024/0052346 A1
(43) Pub. Date: Feb. 15, 2024

(54) METHODS FOR PRODUCING EXTRACELLULAR VESICLES ENRICHED IN ANTI-INFLAMMATORY MICRORNAS

(71) Applicant: Cellular Biopharma(Shanghai) Ltd., Shanghai (CN)

(72) Inventors: Ping LI, Shanghai (CN); Jing WANG, Shanghai (CN); Meiping SHEN, Shanghai (CN); Chengxiang DAI, Shanghai (CN)

(21) Appl. No.: 18/259,603

(22) PCT Filed: Dec. 29, 2021
(86) PCT No.: PCT/CN2021/142539

§ 371 Date: Jun. 28, 2023

(30) Foreign Application Priority Data
Dec. 30, 2020 (CN) 202011612853.2

(51) Int. Cl. C12N 15/113; C12N 5/0775; C12N 5/00; A61P 29/00

(52) U.S. Cl. CPC C12N 15/113; C12N 5/0662; C12N 5/0018; A61P 29/00; B82Y 5/00

(57) ABSTRACT

Provided are methods of producing exosomes by culturing isolated stem cells with an effective amount of at least one immunoregulatory factor. The thus-produced exosomes are enriched in anti-inflammatory miRNAs and/or miRNAs that inhibits the a epithelial-mesenchymal transition.

### CROSS REFERENCE TO RELATED APPLICATION

The present application claims priority to Chinese patent application No. 2020116128532, filed on Dec. 30, 2020, which is hereby incorporated by reference in its entirety.

### SEQUENCE LISTING

This application contains a Sequence Listing which has been filed electronically in ASCII format and is hereby incorporated by reference in its entirety. Said ASCII copy, created on Dec. 23, 2021, is named 11299-009944-WO0_ST25.txt and is 2 KB in size.

### TECHNICAL FIELD

The present disclosure relates to the field of biotechnology, and in particular to methods for increasing the level of specific miRNAs in extracellular vesicles derived from human cells.

### BACKGROUND

Extracellular vesicles (EVs) are cell-derived membranous structures which contain a phospholipid bilayer. Depending on their size and mechanism of biogenesis, they may be classified as exosomes, microvesicles and apoptotic bodies. EVs can also be broadly divided into two categories, ectosomes and exosomes. Exosomes are secreted by cells and play an important role in intercellular signal transduction. The multivesicular body (MVB) is an endosome defined by intraluminal vesicles (ILVs) that bud inward into the endosomal lumen. If the MVB fuses with the cell surface (the plasma membrane), these ILVs are released as exosomes.

The main characteristics of extracellular vesicles may include: 1) a size of 30-150 nm in diameter, 2) having a phospholipid bilayer, which is shown under a transmission electron microscope to be a cup-shaped vesicle structure, and 3) having tetraspanins, such as CD63, CD9, CD81, etc., and markers, such as TSG101, Alix, etc. Exosomes contain constituents (e.g., proteins, DNAs, and RNAs) of the cells that secrete them. RNAs include small RNAs such as microRNAs (miRNAs), long non-coding RNAs (long ncRNAs, lncRNAs), circular RNAs (circRNAs), etc. They are taken up by distant cells, where they can affect cell function and behavior. Intercellular communication through exosomes seems to be involved in the pathogenesis of various disorders, including cancer, neurodegeneration, and inflammatory diseases. Kalluri et al., The biology, function, and biomedical applications of exosomes, Science, 2020, 367 (6478):eaau6977.

Inflammation is a complicated biological and pathophysiological cascade of responses to infections and injuries, and inflammatory mechanisms are closely related to many diseases. The magnitude, the complicated network of pro- and anti-inflammatory factors, and the direction of the inflammatory response can impact on the development and progression of various disorders. The currently available treatment strategies often target the symptoms and not the causes of inflammatory disease and may often be ineffective.

MicroRNAs (miRNAs) play an important role in cell activities such as proliferation and apoptosis. They have also emerged as key gene regulators to control inflammation. They are fine-tune signaling regulators to allow for proper resolution and prevent uncontrolled progress of inflammatory reactions. Tahamtan et al., Anti-Inflammatory MicroRNAs and Their Potential for Inflammatory Diseases Treatment, Front. Immunol. 2018; 9: 1377. Studies have shown that they are involved in the progression of lung inflammation and are useful in the treatment of pulmonary fibrosis. Exosomes derived from mesenchymal stem cells are rich in microRNAs, and may have an anti-inflammatory effect by regulating inflammatory pathways. Increasing the level of certain microRNAs in extracellular vesicles derived from human mesenchymal stem cells is of great significance in treating inflammatory and fibrotic diseases. However, there is no particularly efficient method for increasing certain specific microRNAs in extracellular vesicles.

The present disclosure provides methods for efficiently increasing the levels of specific miRNAs (e.g., anti-inflammatory miRNAs) in extracellular vesicles derived from human cells.

### SUMMARY

The present disclosure provides a method (in vitro) for increasing the level of specific miRNAs in extracellular vesicles derived from human cells.

The present disclosure provides a method for producing extracellular vesicles enriched in at least one anti-inflammatory miRNA and/or at least one miRNA that inhibits epithelial-mesenchymal transition.

The method may comprise: (a) culturing cells (e.g., stem cells) in a cell culture medium for a period of time to allow release of the extracellular vesicles, where the culture medium comprises an amount of at least one immunoregulatory factor, where the extracellular vesicles are released from the cells (e.g., stem cells) into the cell culture medium, and (b) isolating the extracellular vesicles from the cell culture medium.

In certain embodiments, the amount of the at least one immunoregulatory factor is effective for inducing release of extracellular vesicles enriched in at least one anti-inflammatory miRNA and/or at least one miRNA that inhibits epithelial-mesenchymal transition.

The method may further comprise pre-culturing the stem cells to a confluency ranging from about 60% to about 95%, from about 70% to about 95%, from about 70% to about 90%, at least or about 60%, at least or about 70%, at least or about 80%, at least or about 90%, or from about 75% to about 80%, before step (a).

The at least one anti-inflammatory miRNA and/or at least one miRNA that inhibits epithelial-mesenchymal transition may comprise miR-146a, miR-21, let-7a, or combinations thereof.

The extracellular vesicles may comprise exosomes.

In certain embodiments, one or more of the extracellular vesicles (e.g., exosomes) comprise a higher level of at least one anti-inflammatory miRNA and/or at least one miRNA that inhibits epithelial-mesenchymal transition.

In certain embodiments, the at least one immunoregulatory factor comprises an interferon (e.g., one or more interferon).

In certain embodiments, the one or more immunoregulatory factors comprises an interferon and/or tumor necrosis factor (TNF) such as TNFα.

The interferon may comprise interferon γ (IFNγ). The interferon may comprise one or more interferons.

The interferon may be a type I, type II or type III interferon. Type I interferons include interferon-α, interferon-β, interferon-ε, interferon-κ, and interferon-ω. Type II interferons include interferon-γ.

In certain embodiments, the interferon is selected from: IFN-γ, IFN-α, IFN-β, or combinations thereof.

In certain embodiments, the interferon is IFN-γ and does not contain α- and/or β-type IFN (for example, IFN-α and IFN-β).

The amount of the interferon may range from about 1 ng/ml to about 200 ng/ml, from about 1 ng/ml to about 150 ng/ml, from about 1 ng/ml to about 100 ng/ml, from about 5 ng/ml to about 50 ng/ml, from about 5 ng/ml to about 40 ng/ml, from about 5 ng/ml to about 30 ng/ml, from about 5 ng/ml to about 20 ng/ml, from about 5 ng/ml to about 15 ng/ml, from about 5 ng/ml to about 10 ng/ml, from about 8 ng/ml to about 12 ng/ml, from about 9 ng/ml to about 11 ng/ml, from about 1 ng/ml to about 50 ng/ml, from about 1 ng/ml to about 50 ng/ml, from about 1 ng/ml to about 40 ng/ml, from about 1 ng/ml to about 30 ng/ml, from about 1 ng/ml to about 20 ng/ml, from about 1 ng/ml to about 10 ng/ml, or about 10 ng/ml.

The amount of the interferon may be effective for inducing release of exosomes enriched in at least one anti-inflammatory miRNA and/or at least one miRNA that inhibits epithelial-mesenchymal transition.

The culture medium is suitable for culturing the stem cells.

The extracellular vesicles (e.g., exosomes) may have a diameter ranging from about 30 nm to about 200 nm, from about 50 nm to about 200 nm, from about 30 nm to about 150 nm, from about 50 nm to about 150 nm.

The stem cells may be human stem cells. The stem cells may be mesenchymal stem cells, mammary epithelial stem cells, neural stem cells, or cancer stem cells.

In certain embodiments, a level of the at least one anti-inflammatory miRNA may increase by at least or about 1 fold, at least or about 2 folds, at least or about 3 folds, at least or about 4 folds, at least or about 5 folds, at least or about 6 folds, at least or about 7 folds, at least or about 8 folds, at least or about 9 folds, about 2 folds to about 5 folds, about 2 folds to about 7 folds, about 2 folds to about 6 folds, about 2 folds to about 4 folds, about 2 folds to about 3 folds, or about 2 folds to about 8 folds, compared to a level of the at least one anti-inflammatory miRNA in control stem cells not treated with the interferon.

The stem cells may be cultured for a period of time ranging from about 1 day to about 10 days, from about 1 day to about 8 days, from about 1 day to about 6 days, from about 1 day to about 4 days, from about 1 day to about 3 days, about 1 day, about 2 days, about 3 days, about 4 days, about 5 days, about 6 days, about 7 days, or about 8 days.

In some embodiments, the extracellular vesicles (e.g., exosomes) can be isolated by centrifuging, e.g., ultra-centrifuging or sequential centrifuging, the culture medium containing the extracellular vesicles (e.g., exosomes) to obtain a pellet that includes the extracellular vesicles (e.g., exosomes).

Step (b) of the method may comprise centrifuging the culture medium containing the extracellular vesicles released from the stem cells to obtain a pellet that includes the extracellular vesicles. In certain embodiments, in step (b), the culture medium is centrifuged (e.g., sequentially) at 300 g (optional), 2000 g-3000 g (e.g., 3000 g), 10,000 g, and 110,000 g-120,000 g (e.g., 120,000 g).

In certain embodiments, the step of isolating the extracellular vesicles from the cell culture medium (e.g., step (b)) comprises: mixing the cell culture medium with polyethylene glycol (PEG) and incubating for a period of time (e.g., overnight); and separating the mixture (e.g., by centrifuging) to obtain a precipitate, where the precipitate contains the extracellular vesicles.

The present disclosure provides for extracellular vesicles (e.g., exosomes) prepared by the present method.

Also encompassed by the present disclosure is a pharmaceutical composition comprising the extracellular vesicles (e.g., exosomes).

In some embodiments, the composition is cell-free.

The composition can further contain a physiologically acceptable excipient.

The composition may be administered to a subject systemically or locally to a site in need thereof. The composition can contain extracellular vesicles (e.g., exosomes) derived or released from the stem cells obtained from the subject or another subject.

The present disclosure provides for a kit comprising the extracellular vesicles (e.g., exosomes).

The present disclosure also provides for a method of treating a disease in a subject. The method may comprise administering the present pharmaceutical composition to the subject.

The disease may be an inflammatory disease, a fibrotic disease, or a combination thereof.

In the first aspect of the present disclosure, a method for increasing the level of specific miRNAs in extracellular vesicles is provided, comprising the following steps:
(a) providing a stem cell;
(b) under suitable culturing conditions, pre-culturing the stem cell, to obtain a pre-cultured stem cell with a confluence ranging between 60-95%;
(c) in a culture medium containing one or more immunoregulatory factors, inducing and culturing the pre-cultured stem cell, to obtain the culture medium which contains the extracellular vesicles released from the stem cell; wherein the one or more immunoregulatory factors include an interferon; and
(d) isolating the extracellular vesicles from the culture medium.

In certain embodiments, in the extracellular vesicle, the level of one or more of the following miRNAs is increased: miR-146a, miR-21, let-7a, or combinations thereof.

In certain embodiments, the extracellular vesicle has one or more of the following characteristics:
(m1) miR-21 (for example, miR-21-5p) with an increased relative expression level M1, where M1≥3, or M1 ranges from 3 to 6;
(m2) miR-146a (for example, miR-146a-5p) with an increased relative expression level M2, where M2≥3, or M2 ranges from 3 to 9; and
(m3) let-7a (for example, let-7a-5p) with an increased relative expression level M3, where M3≥3, or M3 ranges from 3 to 6;
where the relative expression level is obtained by comparing with the expression level of the corresponding miRNA in an extracellular vesicle (control) prepared under the same conditions but without treating the stem cell with the one or more immunoregulatory factors (where the level of the miRNA in the control is defined as 1).

In certain embodiments, the isolated extracellular vesicle has one or more of the following characteristics:
(f1) TGFβ1 with an increased relative expression level E1, where E1≥1.5, or E1 ranges from 1.5 or 2.0; and/or
(f2) HGF with an increased relative expression level E2, where E2≥1.5, or E1 ranges from 1.5 or 2.0;
where the relative expression level is obtained by comparing with the expression level of TGFβ1 or HGF in an extracellular vesicle (control) prepared under the same conditions but without the one or more immunoregulatory factors (where the expression level of TGFβ1 or HGF in the control is defined as 1).

In certain embodiments, the extracellular vesicle has:
(f3) MMP8 with a decreased relative expression level E3, where E3≤0.6, or E3 ranges from 0.4 to 0.6;
where the relative expression level is obtained by comparing with the expression level of MMP8 in an extracellular vesicle (control) prepared under the same conditions but without the one or more immunoregulatory factors (where the expression level of MMP8 in the control is defined as 1).

In certain embodiments, the extracellular vesicle has one or more of the following characteristics:
(f4) IL1β with a similar relative expression level E4, where E4≤15, for example, 1-15, or 1-12; and/or
(f5) IL6 with a similar relative expression level E5, where E5≤10, for example, 1-10,or1-6;
where the relative expression level is obtained by comparing with the expression level of IL1β or IL6 in an extracellular vesicle (control) prepared under the same conditions but without the one or more immunoregulatory factors (where the expression level of IL1β or IL6 in the control is defined as 1).

In certain embodiments, the method is used to increase the level of miRNAs with anti-inflammatory activity (or anti-inflammatory miRNAs) in extracellular vesicles.

In certain embodiments, the anti-inflammatory miRNAs are selected from: miR-146a-5p, miR-21-5p, or a combination thereof.

In certain embodiments, the step of isolating the extracellular vesicles from the cell culture medium (e.g., step (d)) comprises:
(d1) separating the culture medium, to obtain the cell-free supernatant, wherein the supernatant contains the extracellular vesicles;
(d2) mixing the supernatant in step (d1) with polyethylene glycol (PEG) and incubating for a period of time (e.g., overnight); and
(d3) separating the mixture obtained in step (d2), to obtain a precipitate, where the precipitate contains the extracellular vesicles.

In certain embodiments, the one or more immunoregulatory factors comprise one or more human immunoregulatory factors.

In certain embodiments, the one or more immunoregulatory factors comprise one or more recombinant immunoregulatory factors.

In certain embodiments, the one or more immunoregulatory factors comprise IFN-γ.

In certain embodiments, the one or more immunoregulatory factors include IFN and TNFα, wherein the mass ratio of TNFα to IFN is about 0.01:1 to about 1:1, or about 0.01:1 to about 0.2:1.

In certain embodiments, the stem cells are mesenchymal stem cells.

In certain embodiments, the mesenchymal stem cells are derived from mammals, such as human.

In certain embodiments, the stem cells are derived from human tissues, such as fat, bone marrow, placenta, or combinations thereof.

In certain embodiments, the stem cells are adipose tissue-derived stem cells.

In certain embodiments, the confluence of the stem cells is 60-95%, 70-90%, or 75-80% after pre-culturing.

In certain embodiments, the culture medium contains platelet lysate or fetal bovine serum (FBS). In certain embodiments, the concentration of the platelet lysate in the culture medium is about 2% (v/v) to about 15% (v/v), or about 5% (v/v) to about 10% (v/v).

In certain embodiments, the culture medium is Minimum Essential Medium α (MEM a or aMEM) containing platelet lysate or fetal bovine serum (FBS).

In certain embodiments, the duration of the induction and culturing is about 24 hours to about 48 hours (e.g., in step (c)).

In certain embodiments, the one or more immunoregulatory factors comprise IFN-γ (for example, SEQ ID NO: 1).

In certain embodiments, the concentration of IFN-γ in the culture medium/system is about 5-40 ng/ml, about 5-20 ng/ml, or about 9-11 ng/ml (calculated based on the volume of the culturing medium/system).

In certain embodiments, in step (d1), the culture medium is centrifuged to obtain a cell-free supernatant.

In certain embodiments, in step (d1), the centrifuging is about 3,000-10,000 g differential centrifugation for about 10-40 min.

In certain embodiments, in step (d2), the polyethylene glycol (PEG) is selected from PEG4000-PEG8000, such as PEG6000.

In certain embodiments, the concentration of PEG (e.g., PEG6000) in the mixture is about 8% (w/v) to about 20% (w/v), about 8% (w/v) to about 18% (w/v), about 5% (w/v) to about 20% (w/v), about 8% (w/v) to about 16% (w/v), about 8% (w/v) to about 15% (w/v), about 8% (w/v) to about 12% (w/v), about 8% (w/v), about 9% (w/v), about 10% (w/v), about 11% (w/v), about 12% (w/v), about 13% (w/v), about 14% (w/v), about 15% (w/v), about 16% (w/v), about 7% (w/v), or about 10% (w/v) to about 12% (w/v).

In certain embodiments, in step (d3), the culture medium is centrifuged to obtain a precipitate, where the precipitate contains the extracellular vesicles.

In certain embodiments, in step (d3), the centrifuging is about 3,000-15,000 g differential centrifugation for about 40-70 min.

In certain embodiments, the method further comprises step (e): measuring the miRNA level, cell viability and marker expression level of the extracellular vesicles obtained in step (d).

In certain embodiments, measuring the marker expression level comprises measuring the gene expression level (e.g., mRNA level) and/or the protein level of the markers.

In certain embodiments, the markers include growth factors, proinflammatory factors, and extracellular vesicle specific markers.

In certain embodiments, the growth factors are selected from: transforming growth factor β1 (TGFβ1), hepatocyte growth factor (HGF), or a combination thereof.

In certain embodiments, the proinflammatory factors are selected from: interleukin 1β (IL1β), interleukin 6 (IL6), metalloproteinase 8 (MMP8), or combinations thereof.

In certain embodiments, the extracellular vesicle specific markers are selected from CD9, CD63, CD81, or combinations thereof.

In certain embodiments, the miRNAs include miR-146a, miR-21, let-7a, or combinations thereof.

In certain embodiments, the miR-146a is miR-146a-5p and/or miR-146a-3p.

In certain embodiments, the miR-21 is miR-21-5p and/or miR-21-3p.

In certain embodiments, the let-7a is let-7a-5p and/or let-7a-3p.

In the second aspect of the present disclosure, an extracellular vesicle with an increased level of one or more miRNAs (e.g., anti-inflammatory miRNAs) is provided, where the extracellular vesicle is obtained by the method described in the first aspect of the present disclosure.

In certain embodiments, the extracellular vesicle is derived from human cells.

In certain embodiments, the human cells are induced stem cells and/or mesenchymal stem cells.

In certain embodiments, compared with the extracellular vesicle obtained from the control stem cell not treated with one or more immunoregulatory factors, the present extracellular vesicle has one or more of the following characteristics:
(1) an increased level of one or more anti-inflammatory miRNAs and/or an increased level of one or more miRNAs that inhibit epithelial-mesenchymal transition;
(2) an increased level of the mRNA and/or proteins of growth factors, wherein the growth factors are selected from: TGFβ1, HGF, or a combination thereof;
(3) a decreased level of the mRNA and/or protein of the metalloproteinase 8 (MMP8) (which is proinflammatory); and
(4) a similar or an increased level of extracellular vesicle specific markers.

In certain embodiments, the miRNAs include miR-146a-5p, miR-21-5p, and let-7a-5p.

In certain embodiments, the growth factors include transforming growth factor R 1 (TGFβ1) and/or hepatocyte growth factor (HGF).

In certain embodiments, in the extracellular vesicle, the levels of the mRNA and/or proteins of interleukin 1β (IL1β) and interleukin 6 (IL6) are similar to those of the control extracellular vesicle.

In certain embodiments, the extracellular vesicle specific markers include CD9, CD63 and/or CD81.

In certain embodiments, in the “negative control group,” stem cells are induced and cultured under the same culturing conditions but without the one or more immunoregulatory factors, and the extracellular vesicle is isolated.

In the third aspect of the present disclosure, a pharmaceutical composition is provided, comprising:
(Z1) the extracellular vesicle described in the second aspect of the present disclosure; and
(Z2) a pharmaceutically acceptable carrier.

In certain embodiments, the dosage form of the pharmaceutical composition is selected from: liquid dosage form, or solid dosage form (such as lyophilized dosage form).

In certain embodiments, the pharmaceutical composition is used for treating an inflammatory disease and/or fibrotic disease.

In certain embodiments, the fibrotic diseases include pulmonary fibrosis.

In certain embodiments, the pharmaceutical composition may be an injection or an atomized inhalation preparation.

In the fourth aspect of the present disclosure, a kit, which comprises the pharmaceutical composition described in the third aspect of the present disclosure, is provided.

In certain embodiments, the kit further comprises other drugs for treating an inflammatory disease or fibrotic disease.

In the fifth aspect of the present disclosure, a method for treating a disease is provided, comprising: administering the extracellular vesicle of the second aspect of the present disclosure to a subject in need thereof.

In certain embodiments, the disease is selected from: an inflammatory disease, a fibrotic disease, or a combination thereof.

In certain embodiments, the disease is selected from: pulmonary fibrosis, pneumonia, respiratory tract inflammation, ARDS, or COPD.

In certain embodiments, the pharmaceutical composition is used to treat a disease associated with, or caused by, a virus.

In certain embodiments, the viruses are selected from: influenza viruses, SARS coronavirus, SARS-Cov-2, and MERS coronavirus.

In certain embodiments, the inflammatory diseases are selected from: viral infectious inflammation, bacterial infectious inflammation, fungal infectious inflammation, autoimmune response inflammation, or combinations thereof.

In certain embodiments, the pharmaceutical composition is used to treat an injury.

The injury is selected from: ischemic injury, hypoxic injury, chemical injury, physical injury, or combinations thereof.

It should be understood that, within the scope of the present disclosure, the above technical features of the present disclosure and the technical features described in detail below (such as the embodiments) may be combined with each other to form a new or preferred technical solution. Due to the space limitations, it will not be detailed here.

### BRIEF DESCRIPTION OF THE DRAWINGS

FIG. 1 shows the effect of different immunoregulatory factors on the viability of adipose-derived mesenchymal stem cells at passage 4 (P4). The control is a negative control group. TNFα: tumor necrosis factor alpha; IFNγ: interferon 7. TNFα+IFNγ means the two factors were used in combination. Statistical analysis was performed on all the groups and compared with the negative control group. The Cell Counting Kit-8 (CCK8) was used to measure the viability of the cells after 48 hours of culturing in solution B which contained the indicated factors. The results show that the cell viability of the TNFα and the IFNγ groups did not decrease significantly compared with that of the negative control group; and the cell viability of the TNFα+IFNγ group decreased compared with that of the negative control group.

FIGS. 2A-2E show the expression of TGFβ1, HGF, MMP8, IL6 and IL13 at the mRNA level in adipose-derived mesenchymal stem cells at P4 where the cells were or were not treated with different immunoregulatory factors. FIG. 2A shows the expression of the transforming growth factor β1 (TGFβ1) at the mRNA level; FIG. 2B shows the expression of the hepatocyte growth factor (HGF) at the mRNA level; FIG. 2C shows the expression of metalloproteinase 8 (MMP8) at the mRNA level; FIG. 2D shows the expression of interleukin 6 (IL6) at the mRNA level; and FIG. 2E shows the expression of interleukin 1β (IL1β) at the mRNA level. The control is a negative control group. TNFα: tumor necrosis factor alpha; IFNγ: interferon 7. TNFα+IFNγ means the two factors were used in combination. Statistical analysis was performed on all the groups and compared with the negative control group, where *P<0.05, **P<0.001, ***P<0.0001; Error bars, S.D. (standard deviation).

FIGS. 3A-3C show the levels of miR-21-5p, let-7a-5p and miR-146a-5p in the extracellular vesicles derived from adipose-derived mesenchymal stem cells at P4 where the cells were or were not treated with different immunoregulatory factors. FIG. 3A shows the miR-21-5p levels; FIG. 3B shows the let-7a-5p levels; and FIG. 3C shows a comparison of miR-146a-5p levels. The control is a negative control group. TNFα: tumor necrosis factor alpha; IFNγ: interferon 7. TNFα+IFNγ means the two factors were used in combination. Statistical analysis was performed on all the groups and compared with the negative control group, where *P<0.05, **P<0.001, ***P<0.0001; Error bars, S.D.

FIGS. 4A-4F show the nanoparticle tracking analysis (NTA) of the particle concentration and particle size of the extracellular vesicles after being treated with different immunoregulatory factors. FIG. 4A shows the particle size distribution of the negative control group; FIG. 4B shows the particle size distribution of the TNFα group; FIG. 4C shows the particle size distribution of the IFNγ group; FIG. 4D shows the particle size distribution of the TNFα+IFNγ group; FIG. 4E shows the particle density/concentration of the extracellular vesicles; FIG. 4F shows the particle size of the extracellular vesicles. Statistical analysis was performed on all the groups and compared with the negative control group, where *P<0.05, **P<0.001, ***P<0.0001; Error bars, S.D.

FIG. 5 shows the expressions of the markers CD9, CD63 and CD81 of exosomes from adipose-derived mesenchymal stem cells at P4 after being treated with different immunoregulatory factors (samples had the same volume). Ctrl: negative control group; TNF10: TNFα 10 ng/ml; IFN10: IFNγ 10 ng/ml; T+I10: TNFα 10 ng/ml+IFNγ 10 ng/ml.

### DETAILED DESCRIPTION

This disclosure provides methods of producing exosomes by culturing isolated stem cells with an effective amount of at least one immunoregulatory factor. The thus-produced exosomes are enriched in anti-inflammatory miRNAs and/or at least one miRNA that inhibits the epithelial-mesenchymal transition.

The present disclosure provides for in vitro methods for increasing the level of certain miRNAs in extracellular vesicles derived from human cells. Specifically, stem cells are cultured in the presence of one or more immunoregulatory factors (for example, interferon γ), and extracellular vesicles are isolated from the cell culture medium where the extracellular vesicles contain an increased level of anti-inflammatory miRNAs and/or a decreased level of pro-inflammatory factors. In certain embodiments, when mesenchymal stem cells are cultured by the method of the present disclosure, the levels of miR-146a, miR-21 and let-7a in the extracellular vesicle increase significantly, while the levels of pro-inflammatory factors such as interleukin 10 (IL13), interleukin 6 (IL6), metalloproteinase 8 (MMP8), etc. decrease significantly.

In certain embodiments, a population of extracellular vesicles enriched for anti-inflammatory miRNAs and/or at least one miRNA that inhibits the epithelial-mesenchymal transition comprises at least or about 0.1%, at least or about 0.5%, at least or about 1%, at least or about 2%, at least or about 5%, at least or about 10%, at least or about 15%, at least or about 20%, at least or about 25%, at least or about 30%, at least or about 40%, at least or about 50%, at least or about 60%, at least or about 70%, at least or about 75%, at least or about 80%, at least or about 85%, or at least or about 90%, extracellular vesicles that comprise a higher level of one or more anti-inflammatory miRNAs and/or one or more miRNAs that inhibits the epithelial-mesenchymal transition, compared with extracellular vesicles derived from stem cells not treated with the one or more immunoregulatory factors.

The level of the one or more anti-inflammatory miRNAs and/or one or more miRNAs that inhibits the epithelial-mesenchymal transition in the extracellular vesicles may increase by about 1% to about 100%, about 5% to about 90%, about 10% to about 80%, about 5% to about 70%, about 5% to about 60%, about 10% to about 50%, about 15% to about 40%, about 5% to about 20%, about 1% to about 20%, about 10% to about 30%, at least or about 5%, at least or about 10%, at least or about 15%, at least or about 20%, at least or about 30%, at least or about 40%, at least or about 50%, at least or about 60%, at least or about 70%, at least or about 80%, at least or about 90%, at least or about 100%, about 10% to about 90%, about 12.5% to about 80%, about 20% to about 70%, about 25% to about 60%, or about 25% to about 50%, at least or about 1 fold, at least or about 2 folds, at least or about 3 folds, at least or about 4 folds, at least or about 5 folds, at least or about 6 folds, at least or about 7 folds, at least or about 8 folds, at least or about 9 folds, at least or about 10 folds, at least 1.1 folds, at least 1.2 folds, at least 1.3 folds, at least 1.4 folds, at least 1.5 folds, at least 1.6 folds, at least 1.8 folds, at least 2.5 folds, at least 3.5 folds, at least 5 folds, at least 10 folds, at least 15 folds, at least 20 folds, at least 50 folds, at least 100 folds, from about 2 folds to about 100 folds, from about 1.1 folds to about 10 folds, from about 1.1 folds to about 5 folds, from about 1.5 folds to about 5 folds, from about 2 folds to about 5 folds, from about 3 folds to about 4 folds, from about 5 folds to about 10 folds, from about 5 folds to about 100 folds, from about 10 folds to about 100 folds, from about 10 folds to about 20 folds, from about 20 folds to about 100 folds, from about 20 folds to about 50 folds, from about 30 folds to about 100 folds, from about 50 folds to about 100 folds, from about 70 folds to about 100 folds, compared to the level(s) of the one or more anti-inflammatory miRNAs and/or one or more miRNAs that inhibits the epithelial-mesenchymal transition in the control extracellular vesicles derived from stem cells not treated with one or more immunoregulatory factors.

The level of the mRNA and/or protein level of one or more growth factors (e.g., TGFβ1 and/or HGF) in the extracellular vesicles may increase by about 1% to about 100%, about 5% to about 90%, about 10% to about 80%, about 5% to about 70%, about 5% to about 60%, about 10% to about 50%, about 15% to about 40%, about 5% to about 20%, about 1% to about 20%, about 10% to about 30%, about 50% to about 100%, at least or about 5%, at least or about 10%, at least or about 15%, at least or about 20%, at least or about 30%, at least or about 40%, at least or about 50%, at least or about 60%, at least or about 70%, at least or about 80%, at least or about 90%, at least or about 100%, about 10% to about 90%, about 1²0.5% to about 80%, about 20% to about 70%, about 25% to about 60%, or about 25% to about 50%, at least or about 1 fold, at least or about 2 folds, at least or about 3 folds, at least or about 4 folds, at least or about 5 folds, at least or about 6 folds, at least or about 7 folds, at least or about 8 folds, at least or about 9 folds, at least or about 10 folds, at least 1.1 folds, at least 1.2 folds, at least 1.3 folds, at least 1.4 folds, at least 1.5 folds, at least 1.6 folds, at least 1.8 folds, at least 2.5 folds, at least 3.5 folds, at least 15 folds, at least 20 folds, at least 50 folds, at least 100 folds, from about 2 folds to about 100 folds, from about 1.1 folds to about 10 folds, from about 1.1 folds to about 5 folds, from about 1.5 folds to about 5 folds, from about 2 folds to about 5 folds, from about 3 folds to about 4 folds, from about 5 folds to about 10 folds, from about 5 folds to about 100 folds, from about 10 folds to about 100 folds, from about 10 folds to about 20 folds, from about 20 folds to about 100 folds, from about 20 folds to about 50 folds, from about 30 folds to about 100 folds, from about 50 folds to about 100 folds, from about 70 folds to about 100 folds, compared to the level(s) of the mRNA and/or protein level(s) of one or more growth factors in the control extracellular vesicles derived from stem cells not treated with one or more immunoregulatory factors.

The level of the mRNA and/or protein level of one or more pro-inflammatory factors (e.g., MMP8, IL1β, and/or IL6) in the extracellular vesicles may decrease by about 1% to about 100%, about 5% to about 90%, about 10% to about 80%, about 5% to about 70%, about 5% to about 60%, about 10% to about 50%, about 15% to about 40%, about 5% to about 20%, about 1% to about 20%, about 10% to about 30%, at least or about 5%, at least or about 10%, at least or about 15%, at least or about 20%, at least or about 30%, at least or about 40%, at least or about 50%, at least or about 60%, at least or about 70%, at least or about 80%, at least or about 90%, at least or about 100%, about 10% to about 90%, about 1²0.5% to about 80%, about 20% to about 70%, about 25% to about 60%, about 25% to about 50%, about 40% to about 60%, at least or about 1 fold, at least or about 2 folds, at least or about 3 folds, at least or about 4 folds, at least or about 5 folds, at least or about 6 folds, at least or about 7 folds, at least or about 8 folds, at least or about 9 folds, at least or about 10 folds, at least 1.1 folds, at least 1.2 folds, at least 1.3 folds, at least 1.4 folds, at least 1.5 folds, at least 1.6 folds, at least 1.8 folds, at least 2.5 folds, at least 3.5 folds, at least 15 folds, at least 20 folds, at least 50 folds, at least 100 folds, from about 2 folds to about 100 folds, from about 1.1 folds to about 10 folds, from about 1.1 folds to about 5 folds, from about 1.5 folds to about 5 folds, from about 2 folds to about 5 folds, from about 3 folds to about 4 folds, from about 5 folds to about 10 folds, from about 5 folds to about 100 folds, from about 10 folds to about 100 folds, from about 10 folds to about 20 folds, from about 20 folds to about 100 folds, from about 20 folds to about 50 folds, from about 30 folds to about 100 folds, from about 50 folds to about 100 folds, from about 70 folds to about 100 folds, compared to the level(s) of the mRNA and/or protein level(s) of one or more pro-inflammatory factors in the control extracellular vesicles derived from stem cells not treated with one or more immunoregulatory factors.

In one embodiment, the cell culture medium for mesenchymal stem cells is MEM a (Minimum Essential Medium α).

In another embodiment, the cell culture medium for mesenchymal stem cells is MSC Medium (500 m1 low-glucose DMEM, 25 m1 vesicle-depleted bovine serum, GlutaMAX 1%).

In one embodiment, the cell culture medium for mammary epithelial stem cells is Mammary Epithieal Basal Medium (250 m1 MCDB-170, 250 m1 DMEM-F12, 1.2 g sodium bicarbonate, 2.5 μg EGF, 0.25 mg hydrocortisone, 2.5 mg insulin, 35 mg BPE).

The present disclosure provides a method for preparing extracellular vesicles.

In certain embodiments, the present disclosure provides a method for increasing in vitro the level of miR-146a and miR-21 in extracellular vesicles derived from human cells. The method may comprise the following steps:
(1) in a culture medium (e.g., a culture medium containing a platelet lysate (such as solution B) or fetal bovine serum (FBS)), seeding adipose-derived mesenchymal stem cells (e.g., P2-P4), e.g., at a cell seeding density of about 10,000-15,000 cells/cm², culturing the cells to reach a cell confluence of about 70%-90%;
(2) culturing the cells in a culture medium (e.g., a culture medium containing a platelet lysate (such as solution B) or fetal bovine serum (FBS)) containing one or more immunoregulatory factors (such as human recombinant interferon γ) for about 24 hours to about 48 hours;
(3) collecting the culture medium, obtaining the supernatant after 3,000-10,000 g differential centrifugation of the culture medium; and
(4) after co-incubating the supernatant obtained in step (3) with polyethylene glycol (PEG) (e.g., solution C) e.g., overnight, obtaining the precipitate by 3,000-15,000 g differential centrifugation, which contains extracellular vesicles derived from human cells.

With the method of the present disclosure, the level or expression of certain specific miRNAs (such as miR-146a and miR-21) in extracellular vesicles can be significantly increased, and it does not affect substantially the particle size or the expression of the specific proteins of extracellular vesicles.

The present disclosure provides a method for increasing in vitro the level of certain miRNAs in extracellular vesicles derived from human cells. The method may comprise the following steps: (1) in a medium containing platelet lysate, seeding P2-P4 adipose-derived mesenchymal stem cells at a cell seeding density of about 10,000-15,000 cells/cm², and culturing the cells to a confluence of about 70%-90%; and (2) culturing the stem cells and isolating the extracellular vesicles.

In certain embodiments, the mesenchymal stem cells are mesenchymal stem cells derived from human tissues. The cells may be selected from P2, P3, P4, P5, and P6. In one embodiment, the mesenchymal stem cells are mesenchymal stem cells derived from human tissues, and the cells are selected from P4.

In certain embodiments, the seeding density of the mesenchymal stem cells may be 10,000-15,000 cells/cm², or about 10,000 cells/cm² for pre-culturing or culturing.

In certain embodiments, the concentration of the platelet lysate may be about 2%, 5%, or 10%, in the cell culture medium.

In certain embodiments, the confluence of the mesenchymal stem cells may range from 70%-90%, about 80%, or about 70%.

The present disclosure provides a kit containing a culture medium that can be used to increase the level of certain miRNAs in extracellular vesicles derived from human cells, where the cell culture medium comprises solution B containing the indicated immunoregulatory factors at a concentration of about 10-40 ng/ml.

In certain embodiments, the concentration of the immunoregulatory factor in the cell culture medium is about 10 ng/ml.

In certain embodiments, solution B is a cell culture medium containing about 5% to about 10% (e.g., 10%, or 5%) platelet lysate.

In certain embodiments, solution B is obtained by collecting the supernatant of an aMEM medium containing about 5% to about 10% (e.g., 10%, or 5%) platelet lysate after ultracentrifugation at about 100,000-150,000 g for about 6 hours to about 24 hours.

In certain embodiments, solution B is obtained by collecting the supernatant of an aMEM medium containing about 5% to about 10% (e.g., 10%, or 5%) platelet lysate after ultracentrifugation at about 120,000 g for about 6 hours.

In certain embodiments, polyethylene glycol (PEG) (e.g., solution C) is added to a culture medium, or supernatant containing extracellular vesicles, to a final concentration of about 8% to about 20% PEG (e.g., PEG4000-8000).

The present disclosure provides a method for isolating extracellular vesicles derived from human cells:

In step (3), collecting the supernatant after about 3,000-10,000 g differential centrifugation of the culture medium obtained in step (2); in step (4), co-incubating the supernatant obtained in step (3) with solution C overnight, and obtaining the precipitate by 3,000-15,000 g differential centrifugation, which contains the extracellular vesicles derived from human cells.

In certain embodiments, the culture medium obtained in step (2) is centrifuged at about 3,000 g for about 15 min.

In certain embodiments, the culture medium obtained in step (2) is centrifuged at about 10,000 g for about 30 min.

In certain embodiments, the supernatant obtained in step (3) is co-incubated with PEG (e.g., solution C which contains PEG4000-8000), e.g., overnight, where the final concentration of PEG in the mixture is about 8% to about 20%.

In certain embodiments, the mixture (containing PEG and extracellular vesicles) contains about 12% PEG6000.

In certain embodiments, the supernatant obtained in step (3) is co-incubated overnight with solution C, and then differential centrifugation is performed (at about 3,000 g for about 1 h, or at about 120,000 g for about 70 min).

In certain embodiments, the method further comprises step (5): measuring the cell viability, and measuring the gene expression level (e.g., mRNA level) and/or the protein level of at least one marker.

In certain embodiments, in step (5), the monitoring comprises measuring the gene expression level (e.g., mRNA level) and/or protein level of a marker, wherein the marker may be a positive marker and/or negative marker.

In certain embodiments, positive markers are those whose gene expression level is positively correlated with the extent to increase the level of miR-146a, miR-21 and let-7a in extracellular vesicles derived from human cells. Negative markers are those whose gene expression level is negatively correlated with the extent to increase the level of miR-146a, miR-21 and let-7a in extracellular vesicles derived from human cells.

In certain embodiments, the positive marker is selected from: transforming growth factor β1 (TGFβ1) and hepatocyte growth factor (HGF).

In certain embodiments, the negative marker is selected from: interleukin 1β (IL1β), interleukin 6 (IL6), and metalloproteinase 8 (MMP8).

In certain embodiments, in step (5), the monitoring comprises measuring the relative expression levels of miR-146a, miR-21 and let-7a in extracellular vesicles derived from human cells by fluorescence quantitative PCR.

In certain embodiments, in step (5), the monitoring comprises measuring protein levels of markers CD9, CD63 and CD81 specific to extracellular vesicles derived from human cells by Western blot. This marker is positively correlated with the expression of the protein specific to extracellular vesicles.

In certain embodiments, in step (5), the monitoring comprises measuring the particle concentration and particle size of extracellular vesicles derived from human cells by NTA.

The present disclosure provides that IFNγ can effectively increase the levels of anti-inflammatory miRNAs (such as miR-21-5p and miR146a-5p), and/or miRNAs that inhibit epithelial-mesenchymal transition (such as let-7a-5p) in extracellular vesicles derived from human cells in vitro. The level of the pro-inflammatory factor metalloproteinase 8 (MMP8) is significantly reduced. Therefore, the extracellular vesicles of the present disclosure have better anti-inflammatory activity.

The present methods increase the level (mRNA and/or protein level) of growth factors (e.g., TGFβ1 and HGF) in the extracellular vesicles, which helps to improve the therapeutic effect.

The expression levels of the proteins CD9, CD63 and CD81 specific to the extracellular vesicles obtained in the present disclosure are further increased, which indicates that the extracellular vesicles of the present disclosure have good quality.

Experimental results have shown that the use of culture media containing specific immunoregulatory factors (for example, IFNγ) does not affect cell viability and has low cytotoxic effects.

### Extracellular Vesicles

Extracellular vesicles are membrane enclosed vesicles released by cells. Their primary constituents are lipids, proteins and nucleic acids. They are composed of a lipid-protein bilayer encapsulating an aqueous core comprising nucleic acids and soluble proteins. Extracellular vesicles include, but are not limited to, exosomes, shedding vesicles, microvesicles, small vesicles, large vesicles, microparticles, and apoptotic bodies, based on their size, cellular origin and formation mechanism. Exosomes are formed by inward budding of late endosomes forming multivesicular bodies (MVB) which then fuse with the limiting membrane of the cell concomitantly releasing the exosomes. Shedding vesicles are formed by outward budding of the limiting cell membrane followed by fusion. When a cell undergoes apoptosis, the cell disintegrates and divides its cellular content in different membrane enclosed vesicles termed apoptotic bodies. Non-limiting examples of extracellular vesicles include circulating extracellular vesicles, beta cell extracellular vesicles, islet cell extracellular vesicles, exosomes and apoptotic bodies, and combinations thereof.

There are three major types of extracellular vesicles (EVs), i.e., exosomes, microvesicles, and apoptotic bodies. All of the three major types of EVs are lipid bilayer delimited, and the diameter ranges between 30 and 2,000 nm.

Large extracellular vesicles can range from about 5 μm to about 12 μm in diameter. Apoptotic bodies can range from about 1 μm to about 5 μm in diameter. Microvesicles can range from about 100 nm to about 1 μm in diameter. Exosomes may have a diameter ranging from about 30 nm to about 150 nm, from about 30 nm to about 100 nm, from about 50 nm to about 150 nm, from about 50 nm to about 100 nm, or from about 50 nm to about 200 nm.

Exosomes are small membrane bounded vesicles secreted by almost all types of cells in the cellular microenvironment and are found in biofluids (Lotvall et al., Minimal experimental requirements for definition of extracellular vesicles and their functions: a position statement from the International Society for Extracellular Vesicles, J. Extracell. Vesicles 2014; 3: 26913). They naturally carry biomacromolecules-including different RNAs (mRNAs, regulatory miRNAs), DNAs, lipids, and proteins-and can efficiently deliver their cargoes to recipient cells, eliciting functions, and mediating cellular communications (Thery et al., Exosomes: composition, biogenesis and function, Nat. Rev. Immunol. 2002; 2(8): 569-79).

Exosomes are a main component of paracrine factors of a variety of cells including mesenchymal stem cells (MSCs). MSC exosomes are a type of EVs derived from MSCs. In MSC-derived exosomes, a number of miRNAs and proteins have been identified as changing various activities of the target cells through different pathways. MSC exosomes are involved in physiological and pathological processes such as development, epigenetic regulation, immune regulation (miR-155 and miR-146), tumorigenesis and tumor progression (miR-23b, miR-451, miR-223, miR-24, miR-125b, miR-31, miR-214 and miR-122), etc. According to ExoCarta, an exosome database, over 900 types of proteins have been collected from MSC exosomes. Studies have shown that MSC exosomes contain certain cytokines and growth factors, such as TGFβ1, interleukin-6 (IL-6), IL-10, hepatocyte growth factor (HGF), etc., which have been shown to be helpful in immune regulation. Vascular endothelial growth factor (VEGF), extracellular matrix metalloproteinase inducer (EMMPRIN) and MMP-9 have also been found in MSC exosomes. These proteins play an important role in stimulating angiogenesis and may facilitate tissue repair promoted by exosomes.

In some embodiments, exosomes or extracellular vesicles are derived from cells or tissues including, but not limited to, red blood cells, immune cells, human monocytes and macrophages, tumor cells, epithelial cells, fibroblasts, stem cells (e.g., from primary cells or autologous cells), or bone marrow. Extracellular vesicles or exosomes may be isolated or derived from B cells, T cells, monocytes, or macrophages.

In some embodiments, the exosome or the extracellular vesicle is derived from cultured cells.

In some embodiments, the exosome or extracellular vesicle comprises or contains a cargo or payload. In some embodiments, the cargo or payload is a therapeutic agent. In some embodiments, the cargo or payload is a diagnostic agent.

The cargo or payload of the present exosome or extracellular vesicle includes, but is not limited to, a nucleic acid (DNA, RNA, etc.), a protein, a peptide, a polypeptide, and/or a small molecule.

The disclosure also provides for compositions, including pharmaceutical compositions comprising the exosomes or extracellular vesicles described herein. The disclosure also provides for exosomes or extracellular vesicles described herein for use in treating a disease, and methods of treatment of disease using any of the exosomes or extracellular vesicles described herein.

Methods for isolating extracellular vesicles include size separation methods such as centrifugation. In one embodiment, isolating various components of extracellular vesicles may be through an isolation method including sequential centrifugation. The method may include centrifuging a sample at 800 g for a desired amount of time, collecting the pellet containing cells and cellular debris and (first) supernatant, centrifuging the (first) supernatant at 2,000 g for a desired time, collecting the pellet containing large extracellular vesicles and apoptotic bodies and (second) supernatant. The sequential centrifugation method can further include centrifuging the (second) supernatant at 10,000 g, collecting the pellet containing microvesicles and (third) supernatant. The sequential centrifugation method can further include centrifuging the (third) supernatant at 100,000 g, collecting the pellet containing exosomes (ranging from about 30 nm to about 200 nm in diameter) and (fourth) supernatant. The sequential centrifugation method can further include washing each of the pellets including the extracellular vesicles (e.g., large extracellular vesicles and apoptotic bodies, microvesicles, and exosomes) such as in phosphate buffered saline followed by centrifugation at the appropriate gravitational force and collecting the pellet containing the extracellular vesicles. Isolation, purity, concentration, size, size distribution, and combinations thereof of the extracellular vesicles following each centrifugation step can be confirmed using methods such as nanoparticle tracking, transmission electron microscopy, immunoblotting, and combinations thereof. Nanoparticle tracking (NTA) to analyze extracellular vesicles such as for concentration and size can be performed by dynamic light scattering using commercially available instruments such as ZETAVIEW (commercially available from ParticleMetrix, Meerbusch, Germany). Following isolation, the method can further include detecting an extracellular vesicle marker.

Methods for isolating extracellular vesicles also include using commercially available reagents such as, for example, EXOQUICK TC reagent (commercially available from System Biosciences, Palo Alto, Calif.).

Exosome may be isolated by any suitable techniques, including ultracentrifugation, micro-filtration, size-exclusion chromatography etc. or a combination thereof. Exosome can be isolated using a combination of techniques based on both physical (e.g., size, density) and biochemical parameters (e.g., presence/absence of certain proteins involved in their biogenesis). In certain embodiments, exosomes are isolated using a kit. In one embodiment, exosomes are isolated using the Total Exosome Isolation Kit and/or the Total Exosome Isolation Reagent from Invitrogen.

Following isolation, the method can further include detecting an extracellular vesicle marker of the extracellular vesicle.

Extracellular vesicle or exosome markers include CD9, CD63, CD81, LAPM1, TSG101, and combinations thereof.

### Cells

The cell may a eukaryotic cell. The cell may a mammalian cell, such as a human cell or a non-human mammalian cell (e.g., a non-human primate cell). These include a number of cell lines that can be obtained from American Tissue Culture Collection. In certain embodiments, the cell is infected with a pathogen, e.g., virus, bacteria, mycobacteria, fungi, unicellular organisms. In certain embodiments, the cell is a tumor cell.

The mammal can be a human or a non-human primate. Non-human primates include, but are not limited to, chimpanzees, cynomologous monkeys, spider monkeys, and macaques, e.g., Rhesus. The mammal can be a transgenic non-human mammal.

In certain embodiments, the cell may be removed and maintained in tissue culture in a primary, secondary, immortalized or transformed state. In certain embodiments, the cells are cultured cells or cells freshly obtained from a source (e.g., a tissue, an organ, a subject, etc.). The mammalian cell can be primary or secondary which means that it has been maintained in culture for a relatively short time after being obtained from an animal tissue. These include primary liver cells, primary muscle cells, primary myoblasts, etc.

In certain embodiments, the present method cultures a stem cell or a progenitor cell. Stem cells are undifferentiated cells that have the ability both to self-renew, and to generate differentiated progeny (see Morrison et al. (1997) Cell 88:287-298). In mammals, there are two broad types of stem cells: embryonic stem cells, and adult stem cells, which are found in various tissues.

The stem cells may be bone marrow-derived stem cells (BMSCs), adipose-derived stem cells (ADSCs), neural stem cells (NSCs), blood stem cells, or hematopoietic stem cells. Stem cells can also be from umbilical cord blood. Stem cells may be generated through somatic cell nuclear transfer or dedifferentiation.

The stem cells include, but are not limited to, a blood stem cell, an adipose stem cell, a bone marrow mesenchymal stem cell, a mesenchymal stem cell, a neural stem cell (NSC), a skin stem cell, an endothelial stem cell, a hepatic stem cell, a pancreatic stem cell, an intestinal epithelium stem cell, or a germ stem cell. In certain embodiments, mesenchymal stem cells are isolated from mesodermal organs, such as bone marrow, umbilical cord blood, and adipose tissue.

In certain embodiments, the stem cell is an induced pluripotent stem cell (iPS cell or iPSC). IPSC refers to a type of pluripotent stem cell artificially generated from a non-pluripotent cell, typically an adult somatic cell, or terminally differentiated cell, such as fibroblast, a hematopoietic cell, a myocyte, a neuron, an epidermal cell, or the like.

In certain embodiments, the present method cultures a proliferating cell. In certain embodiments, the present method cultures a T cell (including a primary T cell).

The cells can include autologous cells that are harvested from the subject being treated and/or biocompatible allogeneic or syngeneic cells, such as autologous, allogeneic, or syngeneic stem cells (e.g., mesenchymal stem cells), progenitor cells (e.g., connective tissue progenitor cells or multipotent adult progenitor cells) and/or other cells that are further differentiated.

Any stem cells with differentiation potential can be used in the method for producing induced exosomes, including (but not limited to) embryonic stem cells, induced high-efficacy stem cells, cancer stem cells, and tissue stem cells. The tissue stem cells include, but are not limited to, mesenchymal stem cells, hematopoietic stem cells, mammary stem cells, neural stem cells, small intestinal stem cells, skin stem cells, umbilical cord blood stem cells, limbal stem cells, hair follicle stem cells, adipose tissue derived stem cells, bone marrow stem cells, corneal stem cells, and ovarian stem cells. Stem cells used to produce exosomes can be selected from embryonic stem cells, induced pluripotent stem cells, cancer stem cells, mesenchymal stem cells, hematopoietic stem cells, mammary stem cells, neural stem cells, small intestinal stem cells, skin stem cells, umbilical cord blood stem cells, limbal stem cells, hair follicle stem cells, adipose tissue derived stem cells, bone marrow stem cells, corneal stem cells, and ovarian stem cells.

In certain embodiments of the present method, the cells are contacted with the one or more immunoregulatory factor for a period of time, ranging from about 1 hour to about 30 days, from about 3 hours to about 20 days, from about 5 hours to about 10 days, from about 5 hours to about 5 days, from about 10 hours to about 3 days, from about 12 hours to about 48 hours, from about 12 hours to about 36 hours, about 1 day to about 10 days, about 1 day to about 8 days, about 1 day to about 6 days, about 1 day to about 5 days, about 1 day to about 4 days, about 1 day to about 3 days, about 1 day to about 2 days, about 12 hours, about 1 day, about 2 days, about 3 days, about 4 days, about 5 days, about 6 days, about 7 days, about 8 days, about 9 days, about 10 days, or about 48 hours.

### MicroRNAs

MicroRNAs (miRNAs or miRs) are a class of regulatory RNAs that post-transcriptionally regulate gene expression. MiRNAs are evolutionarily conserved, small non-coding RNA molecules of approximately 18 to 25 nucleotides in length. Weiland et al., (2012) RNA Biol. 9(6):850-859. Bartel DP (2009) Cell 136(2):215-233. Each miRNA is able to downregulate hundreds of target mRNAs comprising partially complementary sequences to the miRNAs. MiRNAs act as repressors of target mRNAs by promoting their degradation, or by inhibiting translation. Braun et al. (2013) Adv.Exp. Med. Biol. 768:147-163.

As a way of post-transcriptional regulation of cells, miRNAs play an important role in cell activities such as proliferation and apoptosis. Some miRNAs impact on important negative feedback loops, while others serve to amplify the response of the immune system by depressing inhibitors of the response. miRNAs target signal transduction proteins involved in the initiation of innate immune responses, and the variety of different miRNAs impact on the intensity of the inflammatory response. The miRNAs may be associated with processes that attenuate inflammation.

Studies have shown that miRNAs are closely related to the progression of pulmonary inflammation. For example, LPS stimulation can cause up-regulation of miR-21 which has an anti-inflammatory effect through the NF-κB pathway. TNF-α stimulation can cause up-regulation of miR-146, which can then down-regulate the levels of pro-inflammatory factors such as IL-6, IL-8, etc. Thus, microRNAs play an important feedback regulation role in the progression of inflammation. In in vivo and in vitro idiopathic pulmonary fibrosis experiments, the microRNAs of the let-7 family were inhibited by transforming growth factor TGF-β and HGMA2 which have high expression level in alveolar epithelial cells, so as to regulate the epithelial-mesenchymal transition (EMT). This indicates that the microRNAs of the let-7 family can be useful in treating pulmonary fibrosis. Exosomes derived from mesenchymal stem cells may be rich in microRNAs, including miR-21 and miR146, and may have an anti-inflammatory effect by regulating inflammatory pathways.

Therefore, increasing the levels of miR-21, miR-146 and/or let-7a in extracellular vesicles derived from mesenchymal stem cells is of great significance in treating inflammatory and fibrotic diseases (e.g., pulmonary fibrosis).

The present EVs may offer targeted and effective delivery of the miRNAs to specific sites with low toxicity. The present EVs may protect the miRNAs from the circulatory nucleases and deliver mRNAs intact to the target site.

Anti-inflammatory miRNAs include one or more of the following: miR-21, miR-146 (miR-146a, miR-146b), let-7a, miR-10a, miR-24, miR-124, miR-145, miR-149, miR-155, miR-181 (miR-181a, miR-181b, miR-181c, miR-181d), miR-9, miR-17-3p, miR-31, miR-99b, miR-125b, miR-126, miR-132, miR-142-3p, miR-187, and miR-210, miR-223. Tahamtan et al., Anti-Inflammatory MicroRNAs and Their Potential for Inflammatory Diseases Treatment, Front. Immunol. 2018; 9: 1377.

MiR-10a and its actions are well conserved among vertebrates and found to be an important posttranscriptional mediator in the control of inflammation (Qin et al., Role of microRNAs in endothelial inflammation and senescence, Mol Biol Rep (2012) 39:4509-18). Importantly, its downregulation has been reported in inflammatory disorders such as rheumatoid arthritis (RA), inflammatory bowel disease (IBD), colitis, acute pancreatitis, and atherosclerosis (Fang et al., MicroRNA-10a regulation of proinflammatory phenotype in athero-susceptible endothelium in vivo and in vitro. Proc Natl Acad Sci USA (2010) 107:13450-5; Xue et al., Microbiota downregulates dendritic cell expression of miR-10a, which targets IL-12/IL-23p40. J Immunol (2011) 187:5879-86; Liu et al., Identification of serum microRNAs as diagnostic and prognostic biomarkers for acute pancreatitis, Pancreatology (2014) 14:159-66; Wu et al., miR-10a inhibits dendritic cell activation and Th1/Th17 cell immune responses in IBD. Gut (2015) 64(11):1755-64; Mu et al., A novel NF-κB/YY1/microRNA-10a regulatory circuit in fibroblast-like synoviocytes regulates inflammation in rheumatoid arthritis, Sci Rep (2016) 6:20059). This miRNA is predominantly expressed in the intestines and contributes to the maintenance of intestinal homeostasis as described earlier. MiR-10a inhibits multiple target genes involved in NF-xB signaling and is important in the pathogenesis of inflammatory diseases.

Recent studies have revealed an essential role for miR-21 in the resolution of inflammation by negative feedback of inflammatory pathways (Sheedy et al., Negative regulation of TLR4 via targeting of the proinflammatory tumor suppressor PDCD4 by the microRNA miR-21. Nat. Immunol. (2010) 11:141-7; Feng et al., miR-21 attenuates lipopolysaccharide-induced lipid accumulation and inflammatory response: potential role in cerebrovascular disease, Lipids Health Dis. (2014) 13:27; Lin et al., miR-21 regulates TNF-α-induced CD40 expression via the SIRT1-NF-κB pathway in renal inner medullary collecting duct cells, Cell Physiol. Biochem. (2017) 41:124-36).

The miR-146 family comprises two genes, miR-146a and miR-146b, which are expressed in response to pro-inflammatory stimuli as negative feedback to control excessive inflammation (Kutty et al., Differential regulation of microRNA-146a and microRNA-146b-5p in human retinal pigment epithelial cells by interleukin-1l, tumor necrosis factor-α, and interferon-γ, Mol. Vis. (2013) 19:737-50). Their aberrant expression is associated with various inflammatory disorders such as RA, lupus disease, psoriasis, and osteoarthritis (Xu et al., Association of MicroRNA-146a with autoimmune diseases. Inflammation (2012) 35(4): 1525-9).

The comparison of the measured levels of the one or more miRNAs to a reference amount or the level of one or more of the miRNAs in a control sample can be done by any method known to a skilled artisan. For example, comparing the amount of the microRNA in a sample to a standard amount can include comparing the ratio between 5S rRNA (or the spiked oligonucleotides) and the miRNA in a sample to a published or known ratio between 5S rRNA (or the spiked oligonucleotides) and the miRNA in a control sample.

The level, amount, abundance or concentration of miRNAs may be measured. The measurement result may be an absolute value or may be relative (e.g., relative to a reference oligonucleotide, relative to a reference miRNA, etc.) Measuring or detecting the amount or level of microRNA in a sample can be performed in any manner known to one skilled in the art and such techniques for measuring or detecting the level of an miRNA are well known and can be readily employed. A variety of methods for detecting miRNAs have been described and may include small RNA sequencing (sRNAseq), deep-sequencing, single-molecule direct RNA sequencing (RNAseq), Northern blotting, microarrays, real-time PCR (polymerase chain reaction), reverse transcription PCR (RT-PCR), targeted RT-PCR, in situ hybridization, miRNA Taqman array cards, electrochemical methods (e.g., oxidation of miRNA-ligated nanoparticles), bioluminescent methods, bioluminescent protein reassembly, BRET (bioluminescence resonance energy transfer)-based methods, fluorescence correlation spectroscopy and surface-enhanced Raman spectroscopy (Cissell, K. A. and Deo, S. K. (2009) Anal. Bioanal. Chem., 394:1109-1116).

The methods of the present invention may include the step of reverse transcribing RNA when assaying the level or amount of a miRNA.

Any suitable methods/kits may be used to isolate and assay the level of the miRNA.

There are also commercially available kits, such as the qRT-PCR miRNA Detection Kit available from Ambion, U.S.A., which can be used for detecting and quantifying microRNA using quantitative reverse transcriptase polymerase chain reaction. TaqMan MicroRNA Assays, which employ a target-specific stern-loop reverse transcription primer to compensate for the short length of the mature miRNA, is also available from Applied Biosystems (Life Technologies, Inc., USA). qSTAR MicroRNA Detection Assays, commercially available from OriGene, Inc. (USA), can also be used. U.S. Patent Publication No. 20140024700. Other commercially available kits, such as PAXgene Blood miRNA Kit (which uses silica-based RNA purification technology) can be employed for isolating miRNAs of 18 nucleotides or longer, available from Qiagen, USA. The miScript PCR System, a three-component system which converts miRNA and mRNA into cDNA and allows for detection of miRNAs using SYBR Green-based real-time PCR, can be employed for quantification of mature miRNA, precursor miRNA, and mRNA all from a single sample (also available from Qiagen, USA). GeneCopoeia has a commercial kit available that is based on using RT-PCR in conjunction with SYBR Green for quantitation of miRNA (All-in-One™ miRNA qRT-PCR Detection Kit, available from GeneCopoeia, Inc., USA). mirVANA, available from Life Technologies, Inc. (USA), employs glass fiber filter (GFF)-based method for isolating small RNAs.

The methods for detecting miRNAs can also include hybridization-based technology platforms and massively parallel next generation small RNA sequencing that allow for detection of multiple microRNAs simultaneously. One commercially available hybridization-based technology utilizes a sandwich hybridization assay with signal amplification provided by a labeled branched DNA (Panornics). Another hybridization-based technology is available from Nanostring Technology (nCounter miRNA Expression Assay), where multiple miRNA sequences are detected and distinguished with fluorescently labeled sequence tags. Examples of next-generation sequencing are available from Life Technologies (SOLiD platform) and Illumina, Inc. (e.g., Illumina HumanHT-12 bead arrays).

MiRNAs can be isolated by methods described in the art for isolating small RNA molecules (U.S. Patent Publication No. 20100291580, U.S. Patent Publication No. 20100222564, U.S. Patent Publication No. 20060019258, U.S. Patent Publication No. 20110054009 and U.S. Patent Publication No. 20090023149).

In one embodiment, miRNA may be isolated from a sample by a method comprising the following steps: a) obtaining a sample having an miRNA; b) isolating total RNA from the sample; c) size fractionation of total RNA by, for example, gel electrophoresis (e.g., polyacrylamide gel electrophoresis) to separate RNAs of the appropriate sizes (e.g., small RNAs); d) ligating DNA adapters to one end or both ends of the separated small RNAs; e) reverse transcription of the adapter-ligated RNAs into cDNAs and PCR amplification; and (f) DNA sequencing. Steps (a)-(f) may be conducted in a different order than listed above. Any of the steps (a)-(f) may be skipped or combined.

Other methods for isolation of miRNA from a sample include employing a method comprising the following steps: a) obtaining a sample having an miRNA; b) adding an extraction solution to the sample; c) adding an alcohol solution to the extracted sample; d) applying the sample to a mineral or polymer support; and, e) eluting the RNA containing the miRNA from the mineral or polymer support with an ionic solution. Other procedures for isolating miRNA molecules from a sample can involve: a) adding an alcohol solution to the sample; b) applying the sample to a mineral or polymer solid support; c) eluting miRNA molecules from the support with an ionic solution; and, d) using or characterizing the miRNA molecules. (U.S. Patent Publication No. 20100222564).

MiRNA can also be isolated by methods involving separation of miRNA from mRNA, such as those described in U.S. Patent Publication No. 20060019258. These methods comprise the steps of a) providing a biological isolate including mRNA having a 5′ cap structure and small RNA having a 5′ phosphate; b) contacting the isolate with a phosphate reactive reagent having a label moiety under conditions wherein the label moiety is preferentially added to the 5′ phosphate over the 5′ cap structure, thereby producing labeled small RNA; and c) distinguishing the small RNA from the mRNA according to the presence of the label.

Examples of methods of isolating and/or quantifying microRNAs can also include, but are not limited to, hybridizing at least a portion of the microRNA with a fluorescent nucleic acid (a fluorescent probe), and reacting the hybridized microRNA with a fluorescent reagent, wherein the hybridized microRNA emits a fluorescent light or hybridizing at least a portion of the microRNA to a radio-labeled complementary nucleic acid. There are commercially available products for fluorescent labeling and detection of miRNAs. NCode miRNA Rapid Labeling System and NCode Rapid Alexa Fluor 3 miRNA Labeling System are both commercially available from Life Technologies, Inc. (USA). Furthermore, fluorescent labels are commercially available and can include the Alexa Flour dyes (Molecular Probes), available from Life Technologies, Inc. (USA), Cy dyes (Lumiprobes), the DyLight fluorophores (available from ThermoScientific (USA)), and FluoProbes.

Locked nucleic acid probes can also be employed. For example, the miRCURY LNA microRNA ISH Optimization Kits (FFPE) provides for detection of microRNAs. This kit employs double DIG*-labeled miRCURY LNA™ microRNA Detection that can be used for in situ hybridization and is commercially available from Exiqon (USA and Denmark).

In one embodiment, a probe for detecting a miRNA can include a single-stranded molecule, including a single-stranded deoxyribonucleic acid molecule, a single-stranded ribonucleic acid molecule, a single-stranded peptide nucleic acid (PNA), or a single-stranded locked nucleic acid (LNA). The probe may be substantially complementary, for example 80%, 85%, 90%, 95%, 96%, 97%, 98%, 99% or 100% identical to the complement of the miRNA being detected, such that the probe is capable of detecting the miRNA. In some embodiments, the probe is substantially identical, 80%, 85%, 90%, 95%, 96%, 97%, 98%, 99%, or 100% identical to the miRNA, such that the probe is capable of detecting the complement of the miRNA. In some instances, the probe is at least 5 nucleotides, at least 10 nucleotides, at least 15 nucleotides, at least 20 nucleotides, at least 25 nucleotides, at least 30 nucleotides or at least 40 nucleotides. In some cases, the probe may be no longer than 25 nucleotides, no longer than 35 nucleotides; no longer than 50 nucleotides; no longer than 75 nucleotides, no longer than 100 nucleotides or no longer than 125 nucleotides in length. In some embodiments the probe is substantially complementary to or substantially identical to at least 5 consecutive nucleotides of the miRNA, for example at least 6, 7, 8, 9, 10, 11, 12, 13, 14, 15, 16, 17, 18, 20, 21 and 22, or more consecutive nucleotides. In some embodiments, the probe can be 5-20, 5-25, 5-50, 50-100, or over 100 consecutive nucleotides long.

### Immunoregulatory Factors

As used herein, the immunoregulatory factors are selected from: interferons (such as IFN-γ, IFN-α and IFN-β), a tumor necrosis factor (TNF, e.g., TNFα), or a combination thereof.

Interferons encompasses type I, type II and type III interferons. The interferon may be a human interferon.

Type I interferons include interferon-α, interferon-β, interferon-ε, interferon-κ, and interferon-ω. Type II interferons include interferon-γ. Type III interferons include interferon-λ.

The interferon used in the present methods and compositions may be a peptide or protein having an amino acid sequence substantially identical (e.g., at least 70%, at least 75%, at least 80%, at least 85%, at least 90%, at least 95%, at least 96%, at least 97%, at least 98%, at least 99%, or 100% identical) to all or a portion of the sequence of a wild-type interferon. U.S. Patent Application Nos. 20070274950; 20040247565 and 20070243163; U.S. Pat. Nos. 7,238,344; 6,962,978; 4,588,585; 4,959,314; 4,737,462; 4,450,103; 5,738,845; and PCT Publication No. WO 07/044,083, each of which is incorporated by reference in their entirety.

The interferons may also be modified, such as PEGylated interferons (PEG-IFNs). The interferons that may be used in the present methods also include variants of interferons such as fragments, consensus interferons (CIFNs), interferons with altered glycosylation (non-native glycosylation or aglycosylated), non-natural interferons, recombinant interferons, interferon mutants. Those skilled in the art are well aware of different interferons including those that are commercially available and in use as therapeutics.

Interferons may be synthetic, recombinant or purified. Interferons can also be expressed using a vector that includes a nucleic acid sequence encoding the interferon.

IFNγ is an important immunoregulatory factor in the body, which can promote the processing and presentation of MHC class I and II antigens. IFNγ can induce virus-infected cells to produce a variety of antiviral proteins by binding to cell surface receptors.

In certain embodiments, IFNγ has a molecular weight of 16.8 kDa.

A representative human interferon 7 amino acid sequence is shown in SEQ ID NO: 1:

(SEQ ID NO: 1)
MQDPYVKEAENLKKYFNAGHSDVADNGTLFLGILKN
WKEESDRKIMQSQIVSFYFKLFKNFKDDQSIQKSVE
TIKEDMNVKFFNSNKKKRDDFEKLTNYSVTDLNVQR
KAIHELIQVMAELSPAAKTGKRKRSQMLFQGRRASQ

There may be an optional tag sequence (such as a 6His tag for easy purification) at the N-terminus or C-terminus of the interferon. In addition, the immunoregulatory suppressors (such as interferons) of the present disclosure may include the wild type and the mutant type, wherein the mutant type comprises at least 30%, at least 40%, at least 50%, at least 60%, at least 70%, at least 80%, or at least 90%, of the corresponding biological activity of the wild type.

The concentration of the immunoregulatory factor in the cell culture medium can promote the increase of the level of one or more desired miRNAs and/or reduce the level of one or more pro-inflammatory factors. The concentration of the immunoregulatory factor in the cell culture medium may range from about 0.5 ng/ml to about 200 ng/ml, from about 0.5 ng/ml to about 150 ng/ml, from about 0.5 ng/ml to about 100 ng/ml, from about 1 ng/ml to about 200 ng/ml, from about 1 ng/ml to about 150 ng/ml, from about 1 ng/ml to about 100 ng/ml, from about 1 ng/ml to about 80 ng/ml, from about 1 ng/ml to about 50 ng/ml, from about 1 ng/ml to about 30 ng/ml, from about 1 ng/ml to about 20 ng/ml, from about 1 ng/ml to about 10 ng/ml, from about 5 ng/ml to about 10 ng/ml, about 1 ng/ml to about 10 μg/ml, about 10 ng/ml to about 1 μg/ml, about 10 ng/ml to about 500 ng/ml, about 10 ng/ml to about 250 ng/ml, or about 10 ng/ml to about 100 ng/ml. In certain embodiments, the concentration of the immunoregulatory factor in the cell culture medium is about 10±5 ng/ml.

The concentration of the immunoregulatory factor in the cell culture medium may be at least or about 1 ng/ml, at least or about 2 ng/ml, at least or about 3 ng/ml, at least or about 4 ng/ml, at least or about 5 ng/ml, at least or about 6 ng/ml, at least or about 7 ng/ml, at least or about 8 ng/ml, at least or about 9 ng/ml, at least or about 10 ng/ml, at least or about 11 ng/ml, at least or about 12 ng/ml, at least or about 13 ng/ml, at least or about 14 ng/ml, at least or about 15 ng/ml, at least or about 16 ng/ml, at least or about 17 ng/ml, at least or about 18 ng/ml, at least or about 19 ng/ml, at least or about 20 ng/ml, at least or about 25 ng/ml, at least or about 30 ng/ml, at least or about 35 ng/ml, at least or about 40 ng/ml, at least or about 45 ng/ml, at least or about 50 ng/ml, at least or about 55 ng/ml, at least or about 60 ng/ml, at least or about 65 ng/ml, at least or about 70 ng/ml, at least or about 75 ng/ml, at least or about 80 ng/ml, at least or about 85 ng/ml, at least or about 90 ng/ml, at least or about 95 ng/ml, or at least or about 100 ng/ml. In certain embodiments, the immunoregulatory factor may be present in the culture medium at a concentration of about 10 ng/ml to about 150 ng/ml, about 50 ng/ml to about 150 ng/ml, or about 75 ng/ml to about 150 ng/ml. In certain embodiments, the immunoregulatory factor may be present in the culture medium at a concentration of about 10 ng/ml.

In certain embodiments, the concentration of IFN-γ in the cell culture medium ranges from about 5 ng/ml to about 40 ng/ml, from about 5 ng/ml to about 20 ng/ml, from about 9 ng/ml to about 11 ng/ml (calculated based on the volume of the culturing system/medium).

### Pharmaceutical Compositions

The present disclosure provides a pharmaceutical composition comprising the present exosomes or extracellular vesicles. The present disclosure provides uses of the present exosomes or extracellular vesicles for use in treating a condition or disorder.

In some embodiments, the present exosomes or extracellular vesicles may be mixed with a pharmaceutically acceptable carrier to form a pharmaceutical composition.

Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. In some embodiments, the effective amount alleviates, relieves, ameliorates, improves, reduces the symptoms, or delays the progression of any disease or disorder in the subject. In some embodiments, the subject is a human.

Pharmaceutically acceptable carriers, including buffers, are well known in the art, and may comprise phosphate, citrate, and other organic acids; antioxidants including ascorbic acid and methionine; preservatives; low molecular weight polypeptides; proteins, such as serum albumin, gelatin, or immunoglobulins; amino acids; hydrophobic polymers; monosaccharides; disaccharides; and other carbohydrates; metal complexes; and/or non-ionic surfactants. See, e.g., Remington: The Science and Practice of Pharmacy 20th Ed. (2000) Lippincott Williams and Wilkins, Ed. K. E. Hoover.

The present exosomes or extracellular vesicles may be delivered to a cell by contacting the cell with the exosomes or extracellular vesicles.

The present exosomes or extracellular vesicles or the present composition may be delivered/administered to a subject by any route, including, without limitation, intravenous, intracerebroventricular (ICV) injection, intracisternal injection or infusion, oral, transdermal, ocular, intraperitoneal, subcutaneous, implant, sublingual, subcutaneous, intramuscular, rectal, mucosal, ophthalmic, intrathecal, intra-articular, intra-arterial, sub-arachinoid, bronchial and lymphatic administration. The present composition may be administered parenterally or systemically. The present composition may be administered locally.

Intravenous forms include, but are not limited to, bolus and drip injections. Examples of intravenous dosage forms include, but are not limited to, Water for Injection USP; aqueous vehicles including, but not limited to, Sodium Chloride Injection, Ringer's Injection, Dextrose Injection, Dextrose and Sodium Chloride Injection, and Lactated Ringer's Injection; water-miscible vehicles including, but not limited to, ethyl alcohol, polyethylene glycol and polypropylene glycol; and non-aqueous vehicles including, but not limited to, corn oil, cottonseed oil, peanut oil, sesame oil, ethyl oleate, isopropyl myristate and benzyl benzoate.

The present disclosure provides a pharmaceutical composition, comprising (a) the extracellular vesicle prepared by the method described in the present disclosure; and (b) a pharmaceutically acceptable carrier.

Because the extracellular vesicles of the present disclosure have an increased level of specific miRNAs (such as miR-146a, miR-21 and let-7a), and because the levels of pro-inflammatory factors such as interleukin 1β (IL1β), interleukin 6 (IL6) and metalloproteinase 8 (MMP8) are reduced, the present disclosure is suitable for the treatment of various diseases such as inflammation, fibrosis, infectious diseases, etc.

As used herein, the ingredients of a “pharmaceutically acceptable carrier” are suitable for humans and/or mammals without excessive side effects (such as toxicity, irritation, and allergic reactions). The term “pharmaceutically acceptable carriers” refers to carriers, including various excipients and diluents, used for the administration of therapeutic agents.

The pharmaceutical composition of the present disclosure comprises a safe and effective amount of the active ingredient of the present disclosure and a pharmaceutically acceptable carrier. Such carriers include (but are not limited to): saline, buffer, glucose, water, glycerol, ethanol, and combinations thereof. Generally, a pharmaceutical preparation should match the mode of administration. The dosage form of the pharmaceutical composition of the present disclosure may be an injection, a lyophilized preparation, a stem cell preparation, or an atomized inhalation preparation. For example, it can be prepared by conventional methods with the normal saline or an aqueous solution containing glucose and other adjuvants. It is advisable to prepare the pharmaceutical composition under aseptic conditions.

The effective amount of the active ingredient of the present disclosure may vary with the mode of administration, the severity of the disease to be treated, etc. The preferred effective amount can be determined by those of ordinarily skill in the art based on various factors (e.g., through clinical trials). The factors include but are not limited to, the pharmacokinetic parameters of the active ingredient, such as bioavailability, metabolism, half-life, etc.; the severity of the patient's disease to be treated, the patient's body weight, the patient's immune conditions, the route of administration, etc.

The pharmaceutically acceptable carriers of the present disclosure include (but are not limited to): water, saline, liposomes, lipids, proteins, protein-antibody conjugates, peptides, cellulose, nanogels, or a combination thereof. The selection of the carrier should match the mode of administration, which is well known to those ordinarily skilled in the art.

### Conditions to be Treated

The present disclosure provides methods for treating a disorder in a subject comprising administering to the subject the present composition. The present exosomes or extracellular vesicles may be administered in a therapeutically effective amount. The present exosome or extracellular vesicle may be delivered/administered to a subject for treating a condition, disorder or disease.

The present compositions may be used to treat an inflammatory disease, and/or a fibrotic disorder. The present compositions may be used to treat a viral infection or a bacterial infection.

The present composition may be used in a method to treat a disease such as an inflammatory disease, or an immune disorder such as an autoimmune disorder.

The inflammatory disorder may be an acute inflammatory disorders or chronic inflammatory disorder.

Examples of inflammatory diseases, disorders, or conditions include, without limitation, dermatitis, chronic eczema, psoriasis, multiple sclerosis, rheumatoid arthritis, systemic lupus erythematosus, graft versus host disease, sepsis, diabetes, atherosclerosis, Sjogren's syndrome, progressive systemic sclerosis, scleroderma, acute coronary syndrome, ischemic reperfusion, Crohn's disease, inflammatory bowel disease, endometriosis, glomerulonephritis, myasthenia gravis, idiopathic pulmonary fibrosis, asthma, allergic reaction, acute respiratory distress syndrome (ARDS) or other acute leukocyte-mediated lung injury, vasculitis, or inflammatory autoimmune myositis.

Inflammatory diseases include inflammatory skin diseases such as atopic dermatitis, inflammatory respiratory diseases such as chronic rhinitis, asthma, chronic obstructive pulmonary disease (COPD), and the like, and inflammatory bowel diseases such as ulcerative colitis, Crohn's disease, and the like.

The term “inflammatory disease” as used herein refers to a disease caused by an inflammatory reaction in the mammalian body, and representative examples thereof include respiratory inflammatory diseases such as asthma, chronic obstructive pulmonary disease, rhinitis, and the like; skin inflammatory diseases such as atopic dermatitis, psoriasis, acne, contact dermatitis, and the like; gastrointestinal inflammatory diseases such as gastritis, digestive ulcers, inflammatory bowel disease, and the like; and complications of the above-listed diseases. In addition, inflammatory diseases also include inflammatory reaction-related cancer, for example, lung cancer, stomach cancer, colorectal cancer, and the like.

In addition, the compositions described here are useful in treating conditions which would benefit from rapid resolution of inflammation. Thus, the compositions described here are useful in promoting wound healing, including the healing of burn wounds and diabetic wounds. Other conditions which may be treated according to the methods described here include, chronic pancreatitis, dermatitis, peritonitis, dry eye, bacterial infection, adipose tissue inflammation, localized aggressive periodontitis, temporomandibular joint inflammation, arthritis, postoperative pain, postsurgical cognitive decline, endotoxin shock, HSV-keratitis, allograft rejection, and heart ischemia.

In certain embodiments, the inflammatory disease or disorder is selected from the group consisting of asthma, ischemia reperfusion injury, lyme arthritis, periodontitis, peritonitis, psoriasis, rheumatoid arthritis, scleroderma, and systemic inflammatory response syndrome. In certain embodiments, the inflammatory disease or disorder is selected from the group consisting of oral mucositis, stomatitis, cheilitis, glossitis, and Sjogren's syndrome. In certain embodiments, the inflammatory disease or disorder is osteoarthritis or rheumatoid arthritis. In certain embodiments, the inflammatory disease or disorder is adipose tissue inflammation. In certain embodiments, the inflammatory disease or disorder is vascular inflammation. In certain embodiments, the inflammatory disease or disorder is heart ischemia. In certain embodiments, the inflammatory disease or disorder is endometriosis. In certain embodiments, the inflammatory disease or disorder is oral mucositis. In certain embodiments, the inflammatory disease or disorder is a disease or disorder of the ocular system. In certain embodiments, the disease or disorder of the ocular system is selected from the group consisting of inflammatory diseases of the eye, dry eye syndrome, macular edema and retinopathy. In certain embodiments, the method is a method for promoting corneal wound healing.

In certain embodiments, the method further comprises administering the present composition with an anti-inflammatory agent. In certain embodiments, the compound and the anti-inflammatory agent are contained in the same dosage form, or in separate dosage forms.

In certain embodiments, the disease or disorder is an inflammatory disease or disorder selected from asthma, ischemia reperfusion injury, lyme arthritis, periodontitis, peritonitis, psoriasis, rheumatoid arthritis, scleroderma, oral mucositis, stomatitis, cheilitis, glossitis, Sjogren's syndrome and systemic inflammatory response syndrome. In certain embodiments, the inflammatory disease or disorder selected from asthma, psoriasis, scleroderma, and oral mucositis.

In certain embodiments, the disclosure provides a method of treating a gastrointestinal disease or disorder selected from the group consisting of inflammatory bowel disease (IBD), ulcerative colitis, Crohn's disease, proctitis, pouchitis, Crohn's disease of the pouch, eosinophilic colitis, lymphocytic colitis, collagenous colitis, diversion colitis, chemical colitis, ischemic colitis, infectious colitis, pseudomembranous colitis and indeterminate colitis. In certain embodiments, the disease or disorder is an IBD-related disease or disorder selected from ulcerative colitis, Crohn's disease, proctitis, pouchitis, Crohn's disease of the pouch, eosinophilic colitis, lymphocytic colitis, collagenous colitis, diversion colitis, chemical colitis, and ischemic colitis. In embodiments, the IBD-related disease or disorder is ulcerative colitis or Crohn's disease. In embodiments, the IBD-related disease or disorder is pouchitis. In certain embodiments, the disclosure provides a method of treating a gastrointestinal disease or disorder selected from the group consisting of bowel obstruction, chronic pancreatitis, colitis, colon cancer, congenital gastrointestinal anomalies, gastroschisis, high-output fistula, parenteral nutrition associated liver disease, postoperative ileus (POI), postoperative intestinal inflammation, short bowel syndrome, and sporadic polyposis.

Conditions or diseases to be treated include inflammatory or autoimmune disorders, conditions and diseases, such as inflammatory bowel disease, rheumatoid arthritis, osteoarthritis, psoriatic arthritis, polyarticular arthritis, multiple sclerosis, allergic diseases, psoriasis, atopic dermatitis and asthma, and those pathologies noted above.

The present composition may be used to treat a fibrotic disorder.

Fibrotic disorders may include, but are not limited to, the fibrosis of the liver, gut, kidney, skin, epidermis, endodermis, muscle, tendon, cartilage, heart, pancreas, lung, uterus, nervous system, testis, penis, ovary, adrenal gland, artery, vein, colon, intestine (e.g., small intestine), biliary tract, soft tissue (e.g., mediastinum or retroperitoneum), bone marrow, joint, eye and stomach. In a further particular embodiment, the fibrotic disorder may include liver, kidney, skin, epidermis, endodermis, muscle, tendon, cartilage, heart, pancreas, lung, uterus, nervous system, testis, ovary, adrenal gland, artery, vein, colon, intestine (e.g., small intestine), biliary tract, soft tissue (e.g., mediastinum or retroperitoneum), bone marrow, joint and stomach fibrosis. In a further particular embodiment, the fibrotic disorder may include liver, gut, lung, heart, kidney, muscle, skin, soft tissue, bone marrow, intestinal, and joint fibrosis. In yet another embodiment, the fibrotic disorder may include non-alcoholic steatohepatitis (NASH), pulmonary fibrosis, idiopathic pulmonary fibrosis, skin fibrosis, eye fibrosis (such as capsular fibrosis), endomyocardial fibrosis, mediastinal fibrosis, myelofibrosis, retroperitoneal fibrosis, progressive massive fibrosis (a complication of coal workers' pneumoconiosis), proliferative fibrosis, neoplastic fibrosis, lung fibrosis consecutive to chronic inflammatory airway disease (COPD, asthma, emphysema, smoker's lung, tuberculosis), alcohol or drug-induced liver fibrosis, liver cirrhosis, infection-induced liver fibrosis, radiation or chemotherapeutic-induced fibrosis, nephrogenic systemic fibrosis, Crohn's disease, ulcerative colitis, keloid, old myocardial infarction, scleroderma/systemic sclerosis, arthrofibrosis, some forms of adhesive capsulitis, chronic fibrosing cholangiopathies such as Primary Sclerosing Cholangitis (PSC), Primary Biliary Cholangitis (PBC), biliary atresia, familial intrahepatic cholestasis type 3 (PFIC3), peri-implantational fibrosis and asbestosis.

The terms “fibrosis”, “fibrotic disease”, “fibrotic disorder” and declinations thereof may denote a pathological condition of excessive deposition of fibrous connective tissue in an organ or tissue. More specifically, fibrosis is a pathological process, which includes a persistent fibrotic scar formation and overproduction of extracellular matrix by the connective tissue, as a response to tissue damage. Physiologically, the deposit of connective tissue can obliterate the architecture and function of the underlying organ or tissue.

The fibrosis or fibrotic disorder may be associated with any organ or tissue fibrosis. Illustrative, non-limiting examples of particular organ fibrosis include liver, gut, kidney, skin, epidermis, endodermis, muscle, tendon, cartilage, heart, pancreas, lung, uterus, nervous system, testis, penis, ovary, adrenal gland, artery, vein, colon, intestine (e.g. small intestine), biliary tract, soft tissue (e.g. mediastinum or retroperitoneum), bone marrow, joint or stomach fibrosis. in particular liver, kidney, skin, epidermis, endodermis, muscle, tendon, cartilage, heart, pancreas, lung, uterus, nervous system, testis, ovary, adrenal gland, artery, vein, colon, intestine (e.g. small intestine), biliary tract, soft tissue (e.g. mediastinum or retroperitoneum), bone marrow, joint or stomach fibrosis.

In a particular embodiment, the fibrotic disorder is selected in the group consisting of a liver, gut, lung, heart, kidney, muscle, skin, soft tissue (e.g., mediastinum or retroperitoneum), bone marrow, intestinal, and joint (e.g., knee, shoulder or other joints) fibrosis.

In one embodiment, the fibrotic disorder may be liver, lung, skin, kidney or intestinal fibrosis.

In certain embodiments, fibrotic disorders include: non-alcoholic steatohepatitis (NASH), pulmonary fibrosis, idiopathic pulmonary fibrosis, skin fibrosis, eye fibrosis, endomyocardial fibrosis, mediastinal fibrosis, myelofibrosis, retroperitoneal fibrosis, progressive massive fibrosis (a complication of coal workers' pneumoconiosis), proliferative fibrosis, neoplastic fibrosis, lung fibrosis consecutive to chronic inflammatory airway disease (COPD, asthma, emphysema, smoker's lung, tuberculosis), alcohol or drug-induced liver fibrosis, liver cirrhosis, infection-induced liver fibrosis, radiation or chemotherapeutic-induced fibrosis, nephrogenic systemic fibrosis, Crohn's disease, ulcerative colitis, keloid, old myocardial infarction, scleroderma/systemic sclerosis, arthrofibrosis, some forms of adhesive capsulitis, chronic fibrosing cholangiopathies such as Primary Sclerosing Cholangitis (PSC) and Primary Biliary Cholangitis (PBC), biliary atresia, familial intrahepatic cholestasis type 3 (PFIC3), peri-implantational fibrosis and asbestosis.

The present compositions may be used in conjunction with an anti-inflammatory or analgesic agent such as an opiate agonist, a lipoxygenase inhibitor, such as an inhibitor of 5-lipoxygenase, a cyclooxygenase inhibitor, such as a cyclooxygenase-2 inhibitor, an interleukin inhibitor, such as an interleukin-1 inhibitor, an NMDA antagonist, an inhibitor of nitric oxide or an inhibitor of the synthesis of nitric oxide, a non-steroidal anti-inflammatory agent, or a cytokine-suppressing anti-inflammatory agent, for example with a compound such as acetaminophen, aspirin, codeine, fentanyl, ibuprofen, indomethacin, ketorolac, morphine, naproxen, phenacetin, piroxicam, a steroidal analgesic, sufentanil, sulindac, tenidap, and the like. Similarly, the instant compositions may be administered with an analgesic listed above; a potentiator such as caffeine, an H2 antagonist (e.g., ranitidine), simethicone, aluminum or magnesium hydroxide; a decongestant such as phenylephrine, phenylpropanolamine, pseudoephedrine, oxymetazoline, epinephrine, naphazoline, xylometazoline, propylhexedrine, or levo-desoxyephedrine; an antitussive such as codeine, hydrocodone, caramiphen, carbetapentane, or dextromethorphan; a diuretic; and a sedating or non-sedating antihistamine.

Anti-inflammatory agents include, but are not limited to, prostacyclin, dopamine, ganciclovir, levofloxacin, cimetidine, heparin, antithrombin, erythropoietin, and aspirin.

Examples of other therapeutic agents that may be combined with the present composition, either administered separately or in the same pharmaceutical compositions, include, but are not limited to: (a) VLA-4 antagonists, (b) corticosteroids, such as beclomethasone, methylprednisolone, betamethasone, prednisone, prednisolone, dexamethasone, fluticasone, hydrocortisone, budesonide, triamcinolone, salmeterol, salmeterol, salbutamol, formoterol; (c) immunosuppressants such as cyclosporine (cyclosporine A, Sandimmune®, Neoral®), tacrolirnus (FK-506, Prograf®), rapamycin (sirolimus, Rapamune®) and other FK-506 type immunosuppressants, and mycophenolate, e.g., mycophenolate mofetil (CellCept®); (d) antihistamines (H1-histamine antagonists) such as bromopheniramine, chlorpheniramine, dexchloipheniramine, triprolidine, clemastine, diphenhydramine, diphenylpyraline, tripelennamine, hydroxyzine, methdilazine, promethazine, trimeprazine, azatadine, cyproheptadine, antazoline, pheniramine pyrilamine, astemizole, terfenadine, loratadine, cetirizine, fexofenadine, descarboethoxyloratadine, and the like; (e) non-steroidal anti asthmatics (e.g., terbutaline, metaproterenol, fenoterol, isoetharine, albuterol, bitolterol and pirbuterol), theophylline, cromolyn sodium, atropine, ipratropium bromide, leukotriene antagonists (e.g., zafmlukast, montelukast, pranlukast, iralukast, pobilukast and SKB-106,203), leukotriene biosynthesis inhibitors (zileuton, BAY-1005); (f) non-steroidal anti-inflammatory agents (NSAIDs) such as propionic acid derivatives (e.g., alminoprofen, benoxaprofen, bucloxic acid, carprofen, fenbufen, fenoprofen, fluprofen, flurbiprofen, ibuprofen, indoprofen, ketoprofen, miroprofen, naproxen, oxaprozin, pirprofen, pranoprofen, suprofen, tiaprofenic acid and tioxaprofen), acetic acid derivatives (e.g., indomethacin, acemetacin, alclofenac, clidanac, diclofenac, fenclofenac, fenclozic acid, fentiazac, furofenac, ibufenac, isoxepac, oxpinac, sulindac, tiopinac, tolmetin, zidometacin and zomepirac), fenamic acid derivatives (e.g., flufenamic acid, meclofenamic acid, mefenamic acid, niflumic acid and tolfenamic acid), biphenylcarboxylic acid derivatives (e.g., diflunisal and flufenisal), oxicams (e.g., isoxicam, piroxicam, sudoxicam and tenoxican), salicylates (e.g., acetyl salicylic acid and sulfasalazine) and the pyrazolones (e.g., apazone, bezpiperylon, feprazone, mofebutazone, oxyphenbutazone and phenylbutazone); (g) cyclooxygenase-2 (COX-2) inhibitors such as celecoxib (Celebrex®) and rofecoxib (Vioxx®); (h) inhibitors of phosphodiesterase type IV (PDE IV); (i) gold compounds such as auranofin and aurothioglucose, (j) etanercept (Enbrel®), (k) antibody therapies such as orthoclone (OKT3), daclizumab (Zenapax®), basiliximab (Simulect®) and infliximab (Remicade®), (1) other antagonists of the chemokine receptors, especially CCR5, CXCR2, CXCR3, CCR2, CCR3, CCR4, CCR7, CX3CR1 and CXCR6; (m) lubricants or emollients such as petrolatum and lanolin, (n) keratolytic agents (e.g., tazarotene), (o) vitamin D3 derivatives, e.g., calcipotriene or calcipotriol (Dovonex®), (p) PUVA, (q) anthralin (Drithrocreme®), (r) etretinate (Tegison®) and isotretinoin and (s) multiple sclerosis therapeutic agents such as interferon-beta-lbeta (Betaseron®), interferon (beta-ialpha (Avonex®), azathioprine (Imurek®, Imuran®), glatiramer acetate (Capoxone®), a glucocorticoid (e.g., prednisolone) and cyclophosphamide (t) DMARDS such as methotrexate (u) other compounds such as 5-aminosalicylic acid and prodrugs thereof, hydroxychloroquine; D-penicillamine; antimetabolites such as azathioprine, 6-mercaptopurine and methotrexate; DNA synthesis inhibitors such as hydroxyurea and microtubule disrupters such as colchicine.

The present compositions and methods may be used to treat liver fibrosis, inflammatory conditions of the lung including those caused by viruses such as SARS-CoV-2, bronchitis and sepsis.

### Kits

The present disclosure also encompasses kits containing the present exosomes or extracellular vesicles, or the present composition.

In some embodiments, the kit comprises the present exosomes or extracellular vesicles, or the present composition, and instructions for using the kit. Elements may be provided individually or in combinations.

In some embodiments, a kit comprises one or more reagents for use in a process utilizing one or more of the elements described herein. Reagents may be provided in any suitable container. For example, a kit may provide one or more reaction or storage buffers. Reagents may be provided in a form that is usable in a particular assay, or in a form that requires addition of one or more other components before use (e.g., in concentrate or lyophilized form).

The present disclosure is described further with reference to some embodiments. It should be understood that the embodiments are only used to illustrate the present disclosure but not to limit the scope of the present disclosure. In the following embodiments where no conditions are specified for the experimental method, the conventional conditions are generally used, for example, the conditions described in Sambrook et al., Molecular Cloning: A Laboratory Manual (New York: Cold Spring Harbor Laboratory Press, 1989), or recommendations of the manufacturer. Unless otherwise specified, percentages and parts are weight percentages and parts by weight.

It is to be understood, although not always explicitly stated, that all numerical designations may be preceded by the term “about”. Where particular values are described in the application and claims, unless otherwise stated, the term “about” meaning within an acceptable error range for the particular value should be assumed. As used herein, the term “about” refers to a particular value+10%.

### Experimental Method

(1) Culturing of Human Mesenchymal Stem Cells

Human adipose-derived mesenchymal stem cells at P4 were seeded at 1×10⁴ cells/cm², and the original medium was discarded when they grew to 80%. Solution B (negative control group), solution B containing 10 ng/ml IFNγ, solution B containing 10 ng/ml TNFα, and solution B containing 10 ng/ml TNFα+10 ng/ml IFNγ were added to the cells, respectively, and the cells were cultured at 37° C. with 5% CO₂ for about 48 hours.

(2) Isolation of the Extracellular Vesicles Derived from Human Mesenchymal Stem Cells

The cell culture supernatant was collected and centrifuged at 3000 g for 15 min. The supernatant was used for the next step, and the precipitate was removed. The supernatant was centrifuged at 10,000 g for 0.5 h. The supernatant was then used and cell debris was removed. A solution containing 24% PEG6000 (e.g., solution C) was added into the culture medium (supernatant after centrifugation) to reach a PEG concentration of 12%, and kept overnight at 4° C. It was centrifuged at 3,200 g at 4° C. for 1 hour. The supernatant was discarded, and the precipitate was resuspended in PBS. It was centrifuged at 120,000 g for 70 min. The supernatant was discarded, the precipitate at the bottom of the centrifuge tube was resuspended in PBS.

(3) Measurement of Cell Viability

The CCK8 kit was used to measure the metabolic values of the cells after being cultured for 48 hours in different solution B (with or without the indicated factors).

(4) Measurement of Expression Levels of the Genes by Quantitative Fluorescence PCR (qPCR)

An RNA extraction kit was used to extract the RNA of the cells, and RevertAid™ First Strand cDNA Synthesis Kit was used to reverse-transcribe 1 μg of the RNA. According to the experimental requirements, the probes in TaqMan® Gene Expression assays were used as the primers to measure the mRNA expression levels of the related proteins by quantitative fluorescence PCR. The experiment was repeated three times with GAPDH as the internal control. After the PCR, the Ct values were measured. The 2^{-ΔΔCT} method was used for data analysis in the experiment.

TABLE 1
Primer design for fluorescence quantitative PCR (mRNA)

| Gene | Product ID | Source | Dye |
|---|---|---|---|
| IL 1β | Hs01555410_m1 | Human | FAM-MGB |
| IL 6 | Hs00174131_m1 | Human | FAM-MGB |
| TGFβ1 | Hs00998133_m1 | Human | FAM-MGB |
| IL 10 | Hs00961622_m1 | Human | FAM-MGB |
| HGF | Hs00300159_m1 | Human | FAM-MGB |
| GAPDH | Hs02786624_g1 | Human | VIC-MGB |

Note:
TaqMan Gene Expression Assays were used for qPCR analysis to evaluate the mRNA expression level of related transcription factors.

(5) Measurement of miRNA Levels by Quantitative Fluorescence PCR (qPCR)

An RNA extraction kit was used to isolate miRNA from the extracellular vesicles. NanoDrop was used for quantification. 10 ng was used for the subsequent reverse transcription. TaqMan®Advanced miRNA Assays were used for reverse transcription to cDNA. The experiment was repeated three times with miR-191-5p as the internal control. After the PCR, the Ct values were measured. The 2^{-ΔΔCT} method was used for data analysis in the experiment.

TABLE 2
Primer design for fluorescence quantitative PCR (miRNA)

| Gene | Product ID | Source | Dye |
|---|---|---|---|
| hsa-miR-146a-5p | 478399_mir | Human | FAM-MGB |
| hsa-miR-21-5p | 477975_mir | Human | FAM-MGB |
| hsa-miR-191-5p | 477952_mir | Human | FAM-MGB |
| hsa-let-7a-5p | 478575_mir | Human | FAM-MGB |

Note:
TaqMan ® Advanced miRNA Assays were used for qPCR analysis to evaluate the miRNA expression level.

(6) Measurement of the Expression of the Extracellular Vesicle Markers by Western Blot

The NP-40 lysis buffer with protease inhibitors was used to lyse the exosomes. The BCA kit was used to measure the exosomal protein concentrations. 4× loading buffer was added to a portion of the samples and boiled at 95° C. for 5 min. Samples of the same volume were loaded to electrophoresis gels which were run at 80V for 0.5 h and 120V for 1 h. After the gel electrophoresis, the proteins were transferred to a PVDF membrane at a constant current of 250 mA for 1.5 h. After the transfer, the samples were blocked for 0.5 h. The primary antibodies (anti-CD9, anti-CD63 and anti-CD81) (1:1,000) were added to the blocking buffers, which were incubated at 4° C. overnight. TBST was used to rinse the membrane for 10 min for three times. The secondary antibodies (1:3,000) specific to the primary antibody species were added, and the samples were incubated at room temperature for 3 h. TBST was used to rinse the membrane for 10 min for three times, and horseradish peroxidase (HRP) was used for detection of proteins on Western blots.

(7) Measurement of the Particle Concentration and Particle Size of Extracellular Vesicles by NTA

Deionized water was used to clean the sample cell of the nanoparticle tracking analyzer ZetaView. The instrument was calibrated by polystyrene microspheres. PBS was used to clean the sample cell. The sample was diluted using PBS and the analysis was performed.

### Example 1: Effects of Different Immunoregulatory Factors on the Activities of Adipose-Derived Mesenchymal Stem Cells

After treating the cells with 10 ng/ml of the factors as described above, CCK8 was used to measure the cell viability.

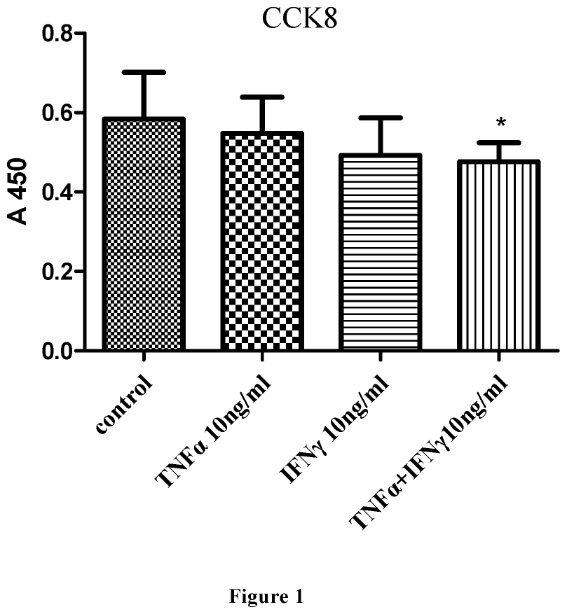

As shown in FIG. 1, viabilities of the cells treated with TNFα or IFNγ did not change significantly compared with that of the negative control group. Viability of the cells treated with TNFα and IFNγ decreased slightly compared with that of the negative control group.

### Example 2: mRNA Expression Levels in Cells Treated with Different Immunoregulatory Factors

After treating the cells with 10 ng/ml of the factors as described above, fluorescence quantitative PCR was used to measure the expression of different mRNAs in the cells.

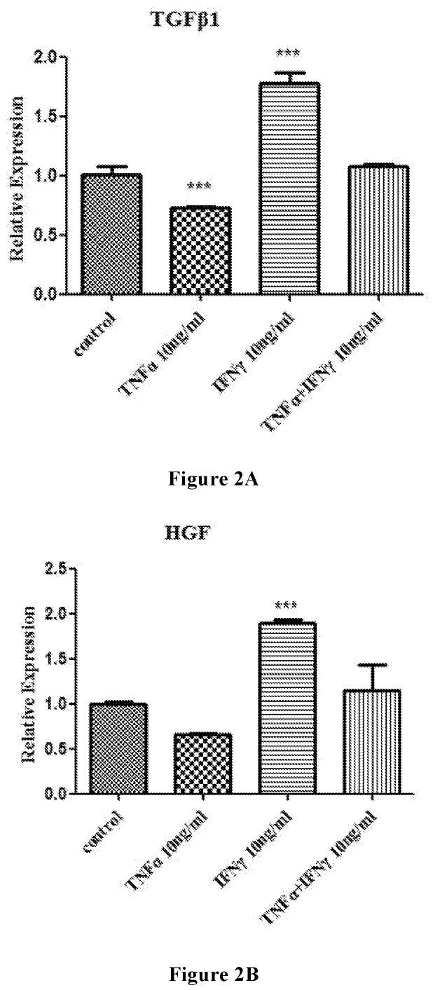

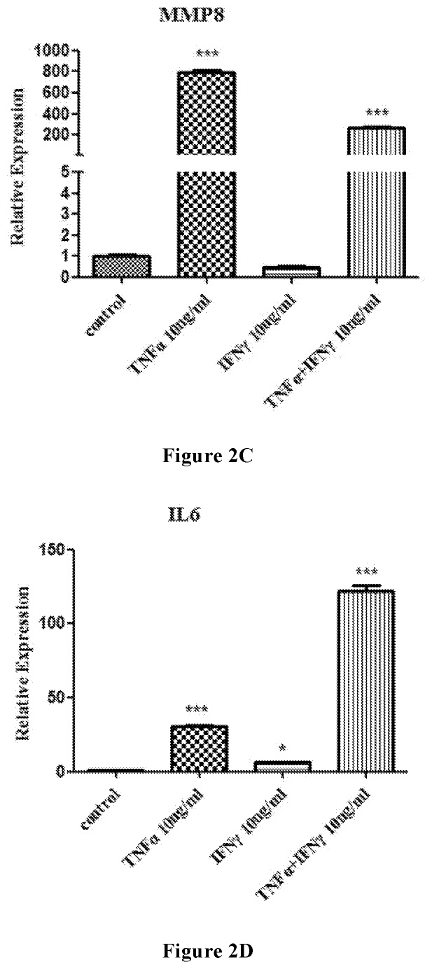

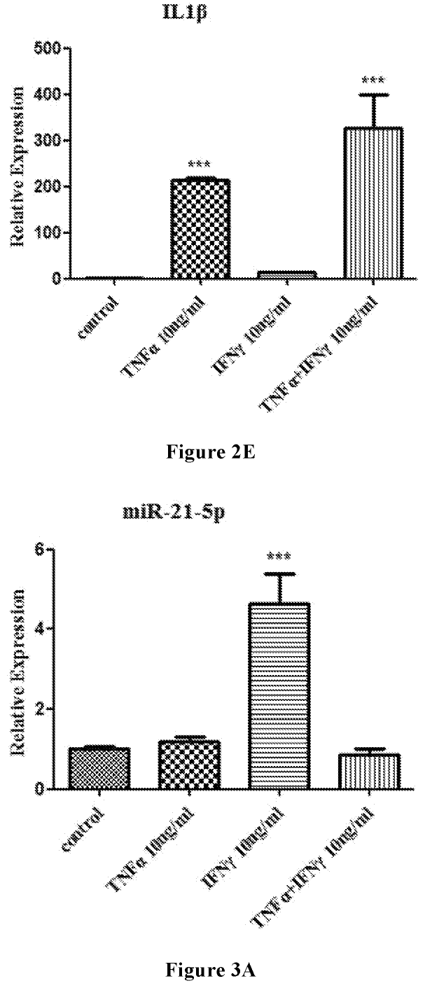

The results are shown in FIGS. 2A-2E.
(a) In cells treated with IFNγ, the mRNA expression levels of transforming growth factor β1 (TGFβ1) and hepatocyte growth factor (HGF) increased significantly compared with those of the negative control group. The mRNA expression level of pro-inflammatory MMP8 decreased significantly compared with that of the negative control group. The mRNA expression levels of IL1β and IL6 increased about several folds (e.g., IL1β increased about 13 folds, and IL6 increased about 6 folds) compared with those of the negative control group, although the increase was much smaller than the increase (by tens to hundreds of folds) when TNFα was added.
(b) In cells treated with TNFα or TNFα+IFNγ, the mRNA expression levels of TGFβ1 and HGF did not increase significantly compared with those of the negative control group. The mRNA expression levels of pro-inflammatory IL1β, IL6 and MMP8 increased significantly, by tens or even hundreds of folds (e.g., IL1β increased more than 200 folds, and IL6 increased more than 30 folds), compared with those of the negative control group.

The above results suggest that, after treatment with specific immunoregulatory factors (e.g., IFNγ), the TGFβ1 and HGF mRNA levels in the cells increased, which led to an increase in TGFβ1 and HGF levels. This resulted in an increase in the level of the mRNA and/or protein of the corresponding proteins in the extracellular vesicles.

After treatment with specific immunoregulatory factors (e.g., IFNγ), the mRNA level of proinflammatory MMP8 decreased which led to a decrease in the synthesis of MMP8 in the cells. This resulted in a decrease in the level of mRNA and/or protein of the factor in the extracellular vesicles.

After treatment with specific immunoregulatory factors (e.g., IFNγ), the mRNA levels of proinflammatory IL1β and IL6 in the cells did not change much, which led to a similar level (or a small increase in the level) of the mRNA and/or protein of IL1β and IL6 in the extracellular vesicles.

### Example 3: miRNA Levels in the Extracellular Vesicles Treated with Different Immunoregulatory Factors

After treating the cells with 10 ng/ml of the factors as described above, an RNA extraction kit was used to extract the RNA from the extracellular vesicles produced from the cells, and the RNA was reverse transcribed to cDNA. The expressions of miR-21-5p, let-7a-5p and miR-146a-5p were measured by qPCR.

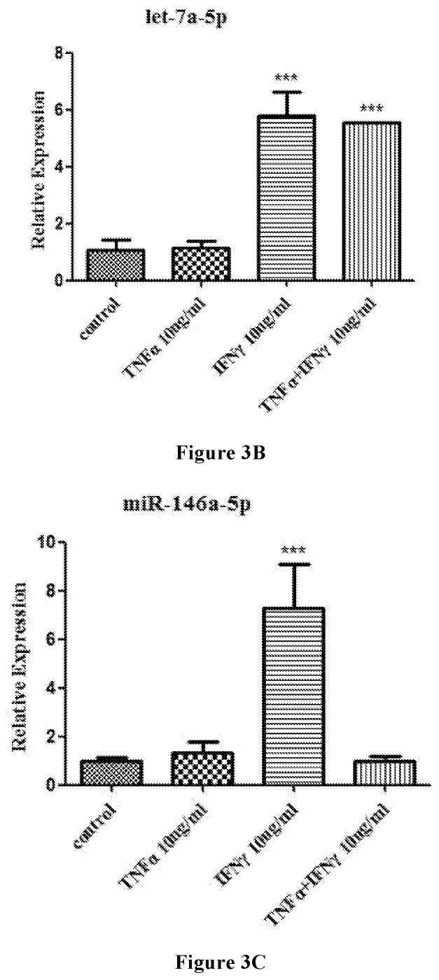

As shown in FIGS. 3A-3C, in cells treated with IFNγ, the expression levels of anti-inflammatory miR-21-5p and miR-146a-5p increased significantly compared with those of the negative control group. The expression level of let-7a-5p, which relates to the regulation of epithelial-mesenchymal transition (EMT), also increased significantly compared with that of the negative control group.

### Example 4: Particle Concentration and Particle Size of the Extracellular Vesicles after Cells were Treated with Different Immunoregulatory Factors Measured by NTA

ZetaView was used to measure the particle concentration and particle size of the extracellular vesicles when cells were treated with different immunoregulatory factors.

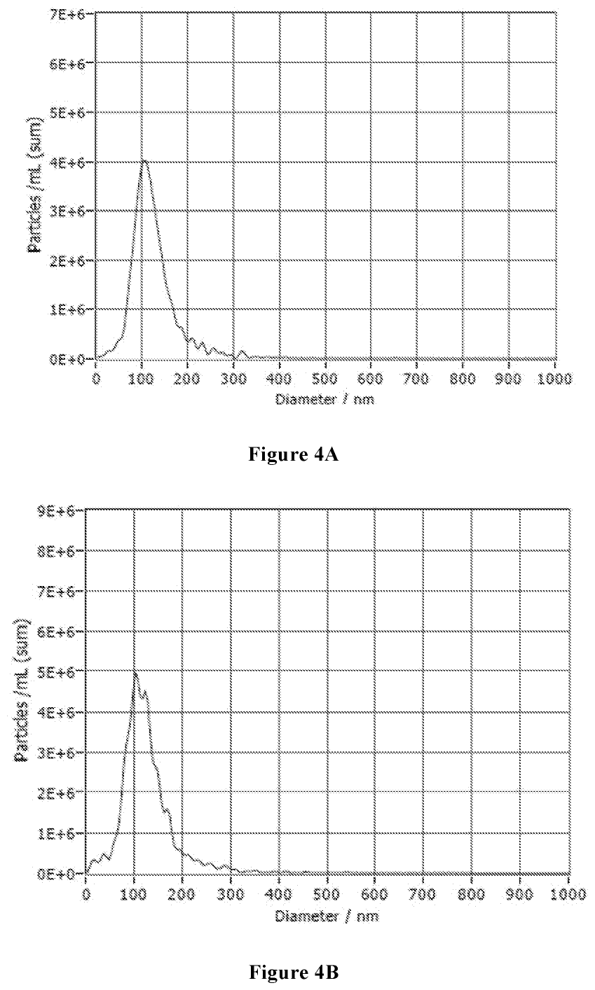

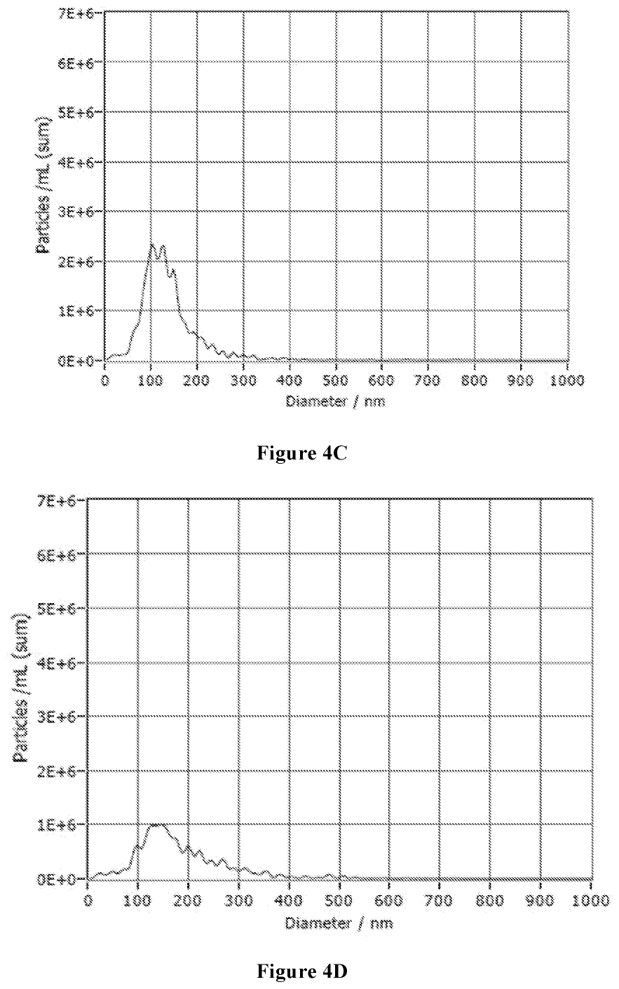

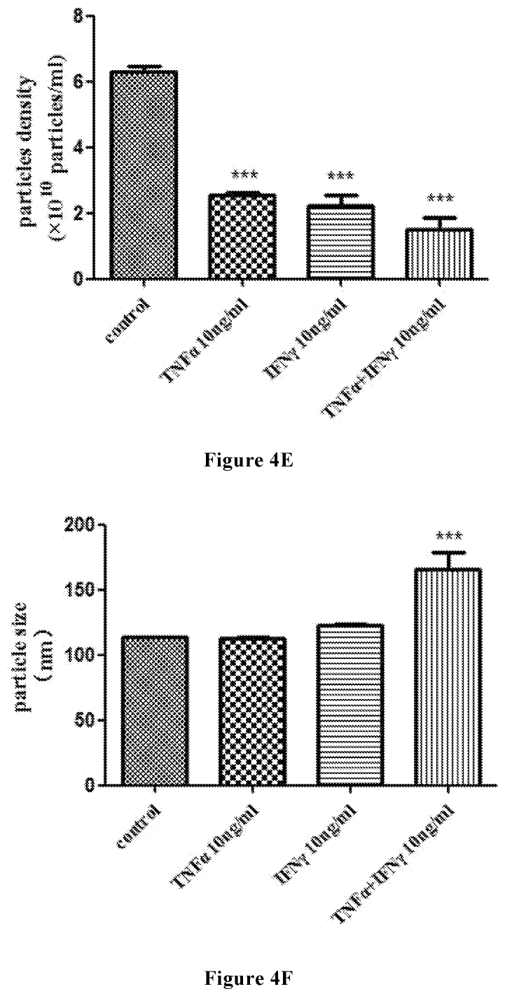

As shown in FIGS. 4A-4F, the particle concentration of all the extracellular vesicles from cells treated with the factors were significantly lower than that of the negative control group. The particle sizes of the extracellular vesicles from cells treated with TNFα or IFNγ did not differ significantly from that of the negative control group. The particle size of the extracellular vesicles from cells treated with TNFα+IFNγ was significantly larger than that of the negative control group.

### Example 5: Expression of Extracellular Vesicle Specific Markers after Cells were Treated with Different Proinflammatory Factors

Western blot was used to measure the expression of the markers specific to extracellular vesicles produced from cells treated with different factors.

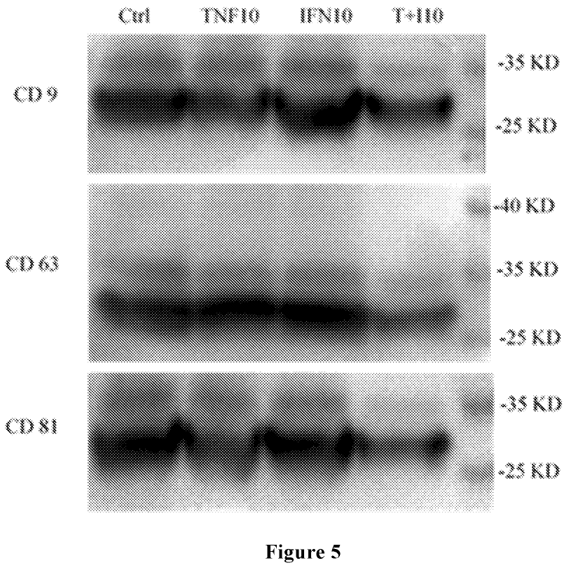

As shown in FIG. 5, the expression levels of CD9, CD63 and CD81 of the extracellular vesicles of the IFNγ group were the highest.

### Discussion

Different culture conditions and induction conditions had different effects on the production of EVs.

The studies show that the levels of miR-146a-5p, miR-21-5p and let-7a-5p of extracellular vesicles from cells cultured in the presence of specific immunoregulatory factors (such as IFNγ) unexpectedly increased significantly compared with those of the negative control group.

As miR-146a-5p and miR-21-5p have anti-inflammatory activity and let-7a-5p has the activity that inhibits epithelial-mesenchymal transition, the EVs generated using the present method have better anti-inflammatory activity and/or the ability to inhibit epithelial-mesenchymal transition (EMT), which is beneficial for the treatment of diseases such as inflammatory diseases or fibrotic diseases.

In addition, after adipose-derived mesenchymal stem cells were treated with IFNγ, the mRNA levels of TGFβ1 and HGF in the cells increased significantly, which led to an increase in the level of the mRNA and/or protein of TGFβ1 and HGF in the extracellular vesicle.

In addition, the mRNA expression of the pro-inflammatory factor MMP8 decreased significantly, while the mRNA levels of IL1β and IL6 did not increase significantly, which is also helpful for the EVs of the present disclosure to be used to treat diseases such as inflammatory diseases or fibrotic diseases.

In addition, the levels of CD9, CD63 and CD81, the extracellular vesicle-specific markers, produced by the secreted exosomes of IFNγ-treated cells were the highest, which suggests that the EVs of the present disclosure have excellent quality.

The treatment with IFNγ, compared with other treatment conditions (such as TNFα, and TNFα+IFNγ), can more effectively increase the levels of the anti-inflammatory miRNAs (e.g., miR-146a-5p and miR-21-5p) in extracellular vesicles and the level of let-7a-5p that inhibits epithelial-mesenchymal transition, and can obtain a favorable cytokine expression profile, thereby contributing to the combined and synergistic effects of the multiple factors (such as TGFβ1 and HGF) and multiple active molecules (miRNAs, mRNAs, and proteins) in the EVs of the present disclosure.

The scope of the present invention is not limited by what has been specifically shown and described hereinabove. Those skilled in the art will recognize that there are suitable alternatives to the depicted examples of materials, configurations, constructions and dimensions. Numerous references, including patents and various publications, are cited and discussed in the description of this invention. The citation and discussion of such references is provided merely to clarify the description of the present invention and is not an admission that any reference is prior art to the invention described herein. All references cited and discussed in this specification are incorporated herein by reference in their entirety. Variations, modifications and other implementations of what is described herein will occur to those of ordinary skill in the art without departing from the spirit and scope of the invention. While certain embodiments of the present invention have been shown and described, it will be obvious to those skilled in the art that changes and modifications may be made without departing from the spirit and scope of the invention. The matter set forth in the foregoing description is offered by way of illustration only and not as a limitation.

1. A method for producing extracellular vesicles enriched in at least one anti-inflammatory miRNA, the method comprising:
(a) culturing stem cells in a cell culture medium for a period of time to allow release of the extracellular vesicles, wherein the culture medium comprises an amount of at least one immunoregulatory factor effective for inducing release of extracellular vesicles enriched in at least one anti-inflammatory miRNA, wherein the extracellular vesicles are released from the stem cells into the cell culture medium, and
(b) isolating the extracellular vesicles from the cell culture medium.

2. The method of claim 1, wherein the at least one immunoregulatory factor comprises an interferon.

3. The method of claim 1, further comprising pre-culturing the stem cells to a confluency ranging from about 60% to about 95% before step (a).

4. The method of claim 1, wherein the at least one anti-inflammatory miRNA comprises miR-146a, miR-21, let-7a, or combinations thereof.

5. The method of claim 2, wherein the interferon comprises interferon γ (IFNγ).

6. The method of claim 2, wherein the interferon has a concentration ranging from about 1 ng/ml to about 50 ng/ml in the cell culture medium.

7. The method of claim 6, wherein the interferon has a concentration of about 10 ng/ml.

8. The method of claim 1, wherein the extracellular vesicles comprise exosomes.

9. The method of claim 1, wherein the extracellular vesicles have a diameter ranging from about 50 nm to about 150 nm.

10. The method of claim 1, wherein the stem cells are human stem cells.

11. The method of claim 1, wherein the stem cells are mesenchymal stem cells, mammary epithelial stem cells, neural stem cells, or cancer stem cells.

12. The method of claim 1, wherein a level of the at least one anti-inflammatory miRNA increases by at least 2 folds compared to a level of the at least one anti-inflammatory miRNA in control stem cells not treated with the at least one immunoregulatory factor.

13. The method of claim 12, wherein a level of the at least one anti-inflammatory miRNA increases by about 2 folds to about 5 folds.

14. The method of claim 12, wherein a level of the at least one anti-inflammatory miRNA increases by about 2 folds to about 8 folds.

15. The method of claim 1, wherein the stem cells are cultured for about 1 day to about 8 days.

16. The method of claim 1, wherein the stem cells are cultured for about 2 days.

17. Extracellular vesicles prepared by the method of claim 1.

18. A pharmaceutical composition comprising the extracellular vesicles of claim 17.

19. A kit comprising the extracellular vesicles of claim 17.

20. A method of treating a disease in a subject, the method comprising administering the pharmaceutical composition of claim 18 to the subject.

21. The method of claim 20, wherein the disease is an inflammatory disease, a fibrotic disease, or a combination thereof.


SEQUENCE LISTING (1 sequence)

SEQ ID NO: 1 - protein, 144 aa, Homo sapiens
MQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFQGRRASQ